(19) United States
(12) Patent Application Publication
(10) Pub. No.: US 2002/0146712 A1
(43) Pub. Date: Oct. 10, 2002

(54) Tumor suppressor

(76) Inventors: Richard D. Goold, Belmont, CA (US); Ingrid E. Akerblom, Redwood City, CA (US); Jeffrey J. Seilhamer, Los Altos Hills, CA (US); Roger Coleman, Mountain View, CA (US)

Correspondence Address: LEGAL DEPARTMENT, INCYTE GENOMICS, INC., 3160 Porter Drive, Palo Alto, CA, 94304, US

(21) Appl. No.: 09/924,654

(22) Filed: Aug. 7, 2001

Related U.S. Application Data
(63) Continuation-in-part of application No. 09/338,353, filed on Jun. 22, 1999, now abandoned.
(63) Continuation-in-part of application No. 08/616,392, filed on Mar. 15, 1996.
(63) Continuation-in-part of application No. 08/581,240, filed on Dec. 29, 1995.

(51) Int. Cl. C12Q 1/68; G01N 33/53; C12P 21/04; C07K 16/28; C07K 14/705

(52) U.S. Cl. 435/6; 435/7.1; 435/70.21; 530/388.22; 530/350

(57) ABSTRACT

The invention provides a cDNA which encodes a tumor suppressor. It also provides for the use of the cDNA, protein, and antibody in the diagnosis, prognosis, treatment and evaluation of therapies for cancer. The invention further provides vectors and host cells for the production of the protein and transgenic model systems.

This application is a continuation-in-part of U.S. Ser. No. 09/338,353 filed Jun. 22, 1999; U.S. Pat. No. 5,998,165, issued Dec. 7, 1999; and U.S. Pat. No. 5,840,870, issued Nov. 24, 1998.

### FIELD OF THE INVENTION

This invention relates to a tumor suppressor, its encoding cDNA, and an antibody which binds the tumor suppressor which can be used in the diagnosis, prognosis, treatment and evaluation of therapies for cancers, particularly of the breast, ovary, pancreas, and prostate.

### BACKGROUND OF THE INVENTION

Tumor suppressors are genetic elements whose loss or inactivation contribute to the deregulation of cell proliferation and the progression of cancer. These genes normally function to control or inhibit cell growth in response to stress and to limit the proliferative life span of the cell. The well known tumor suppressor genes include the genes encoding the retinoblastoma (Rb) protein, p53, and the breast cancer 1 and 2 proteins (BRCA1 and BRCA2). Mutations in these genes are associated with acquired and inherited genetic predisposition to the development of certain cancers.

The role of p53 in the pathogenesis of cancer has been extensively studied and reviewed (Aggarwal et al. (1998) J Biol Chem273:1-4; Levine (1997) Cell 88:323-331). About 50% of all human cancers contain mutations in the p53 gene. These mutations result in either the absence of functional p53 or, more commonly, a defective form of p53 which is overexpressed. p53 is a transcription factor that contains a central core domain required for DNA binding. Most cancer-associated mutations in p53 localize to this domain. In normal proliferating cells, p53 is expressed at low levels and is rapidly degraded. p53 expression and activity is induced in response to DNA damage, abortive nitosis, and other stressful stimuli. In such instances, p53 induces apoptosis or arrests cell growth until the stress is removed. Downstream effectors of p53 activity include apoptosis-specific proteins and cell cycle regulatory proteins, including Rb, cyclins, and cell cycle-dependent kinases.

### Breast Cancer

Breast cancer is the most common cancer affecting women; more than 180,000 new cases are diagnosed each year. The mortality rate for breast cancer approaches 10% of all deaths in females between the ages of 45-54 (Gish (1999) AWIS Magazine 28:7-10). Survival rate varies from 97%, localized breast cancer with early diagnosis, to 22%, for advanced stage, metastatic disease. Classically, breast cancers have been categorized by histologic appearance and location of the lesion. Some of the common categories include adenocarcinoma, ductal carcinoma, lobular carcinoma, in situ carcinoma, infiltrating or invasive carcinoma, and specific inflammatory complications of breast cancer.

Although breast cancer may develop anytime after puberty, it is most common in postmenopausal women and relatively rare in men. The causes and genetic and environmental components of this disease are for the most part unknown; however, many breast cancers are steroid sensitive, and estrogen or androgen may potentiate their growth.

Familial breast cancer accounts for 5% to 9% of known cases and is caused by mutations in two genes, BRCA1 and BRCA2. These diagnostic marker genes not only predispose a subject to breast cancer but may also be passed to offspring (Gish, supra). The vast majority of breast cancers are adenocarcinomas caused by non-inherited mutations in breast epithelial cells. The expression of specific genes associated with breast cancer, for example, epidermal growth factor (EGF) and its receptor, EGFR (a member of the erbB family of proteins) has been well studied. Overexpression of EGFR, particularly coupled with down-regulation of the estrogen receptor, is a marker of poor prognosis. In addition, the elevation of EGFR expression during metastases suggests that EGFR is involved in tumor progression. This is supported by evidence that EGF has effects on cell functions related to metastatic potential, such as cell motility, chemotaxis, secretion and differentiation.

Changes in expression of other members of the erbB receptor family, HER-2/neu, HER-3, and HER-4, and their ligands (Bacus et al. (1994) AmJ Clin Pathol 102:S13-S24) have also been implicated in breast cancer. Other breast cancer diagnostic markers include matrix Gla protein (Chen et al. (1990) Oncogene 5:1391-1395) and maspin (Sager et al. (1996) Curr Top Microbiol Immunol 213:51-64). Each of these markers shows up with some specificity in different forms of breast cancer.

Current procedures for clinical breast examination are lacking in sensitivity and specificity, and efforts are underway to develop comprehensive gene expression profiles for breast cancer that may be used in conjunction with conventional screening methods to improve diagnosis and prognosis (Perou CM et al. (2000) Nature 406:747-752).

### Cancer of the Ovary

Ovarian cancer is the leading cause of death from gynecologic malignancy and the fourth leading cause of cancer death among American women. Since ovarian tumors produce few early signs, the disease is often not identified until its later stages (stage III or IV). About one in 70 women eventually develops ovarian cancer, and one in 100 women dies of it.

Ovarian cancer affects predominantly perimenopausal and postmenopausal women, and incidence of the disease is higher in industrialized countries with a higher dietary fat intake. Familial predisposition to endometrial, breast, or colon cancer increases risk as does nulliparity, infertility, late-childbearing, and delayed menopause; however, the use of oral contraceptives significantly decreases risk (The Merck Manual, 1992, Rahway N.J., Sec 14, Ch 171, pp 1827-1829).

Primary epithelial tumors make up 90% of ovarian cancers and include serous papillary carcinoma, also known as serous cystoadenocarcinoma, mucinous cystoadenocarcinoma, and endometrioid and mesonephric malignancies. Serous papillary carcinomas account for 50% of primary epithelial ovarian cancers.

To date ultrasonography is the method of choice for identification of stage I ovarian cancer, but it is only effective where familial factors, abdominal symptoms, or abnormalities found during routine pap smears raise the need for further examination (Karlan et al. (1999) Am J Obstet Gynecol 180:917-28; Jimenez-Ayala et al. (1996) Acta Cytol 40:765-9). Ovarectomy is the treatment of choice, and peritoneal washing cytology during surgery has been found to be a useful prognostic factor (Suzuki et al. (1999) Oncol Rep 6:1009-12). Confirm metastasis of papillary serous carcinoma is associated with a survival of approximately one year. Since there is only one non-invasive test that women can obtain which will point out the onset of this silent killer, the identification of diagnostic and prognostic markers for ovarian cancer satisfies a need in the art.

### Pancreatic Cancer

Human pancreatic cancer is the fourth most common cause of death in the United States with an incidence in 1993 of 27,700 and a mortality of 24,500 (Caldas et al (1994) Nat Genet 8:27-32). The disease is more common in males than females, and the peak incidence is between 60 and 70 years of age. Incidence of pancreatic cancer is 2-fold greater in patients with diabetes mellitus and about 2.5-fold greater in smokers than non-smokers. The median survival for individuals with pancreatic cancer is six months from diagnosis. About 10% of these patients survive one year, and about 2% survive five years (Harrison Principles of Internal Medicine 11th ed, Braunwald et al McGraw-Hill, New York, N.Y. pg. 1381-1384).

Recently, Schutte et al (1995; Proc Natl Acad Sci 92:5950-5954) used representational difference analysis (as described by Lisitsyn et al (1995) Proc Natl Acad Sci 92:151-155) to demonstrate a homozygous deletion in pancreatic cancer mapping to a 1 cM region at 13ql.3. The deletion was flanked by the markers D135171 and D135260 and found within the 6 cM region identified as containing the BRCA2 locus (600185) of heritable breast cancer susceptibility. Schutte (supra) suggested that the gene might be involved in multiple tumor types and that its function was tumor suppression. Additionally, in 1994, Muller-Pillasch et al. submitted a nucleotide sequence designated g533948 EMBL/Genbank/ DDBJ which was found to be differentially expressed in the pancreatic cancer cell line PATU.

The Sanger Genome Centre (Cambridge, UK) has been involved in a large-scale genonmic DNA sequencing project in the region of human chromosome 13 (13ql2) thought to contain the gene responsible for BRCA2. Sanger recently deposited several large fragments of sequenced DNA on their web site, in unfinished and unannotated form

Current methods for the diagnosis of pancreatic cancer include measurement of serum amylase and lipase values; however, these values are found to be abnormal in only 10% of all pancreatic cancer cases. Standard gastrointestinal X-rays may suggest the presence of carcinoma of the head of the pancreas, but only 50% of all patients with this type of carcinoma have an abnormal examination (Harrison's Principles of Internal Medicine, supra). Also, blood, urine and feces of individuals subject to carcinoma of the body and tail of the pancreas are often normal.

### Prostate Cancer

Prostate cancer is a common malignancy in men over the age of 50, and the incidence increases with age. In the US, there are approximately 132,000 newly diagnosed cases of prostate cancer and more than 33,000 deaths from the disorder each year. Over 95 percent of prostatic cancers are adenocarcinomas which originate in the prostatic acini. The remaining 5 percent are divided between squamous cell and transitional cell carcinomas which arise in prostatic ducts or other parts of the gland.

As with most cancers, prostate cancer develops through a multistage progression ultimately resulting in an aggressive, metastatic phenotype. The initial step in tumor progression involves the hyperproliferation of normal luminal and/or basal epithelial cells that become hyperplastic and evolve into early-stage tumors. Although early-stage tumors are localized in the prostate, they eventually may metastasize and particularly to the bone, brain or lung. About 80% of these tumors are responsive to androgen treatment which controls the growth of prostate epithelial cells. However, in its most advanced state, the cancer becomes androgen-independent and no treatment exists for this condition is known.

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen, and various growth factors, Since PSA levels are not always useful in detecting individual cases of prostate cancer, new and more sensitive compositions are needed.

The discovery of a tumor suppressor, an encoding DNA, and an antibody which binds specifically to the tumor suppressor satisfies a long standing need in the art by providing molecules and compositions which can be used in the diagnosis, prognosis, treatment and evaluation of therapies for multiple cancers, particularly of the breast, ovary, pancreas, and prostate.

### SUMMARY OF THE INVENTION

The present invention is based on the discovery of a tumor suppressor which maps to the same region of chromosome 13 as BRCA2. A cDNA which encodes the tumor suppressor, the protein, and an antibody which specifically binds the protein are useful in the diagnosis, prognosis, treatment and evaluation of therapies for cancers, particularly of the breast, ovary, pancreas, and prostate.

The invention provides an isolated cDNA comprising a nucleic acid sequence of SEQ ID NO:3 which encodes a protein having an amino acid sequence of SEQ ID NO:4. The invention also provides isolated cDNA fragments comprising SEQ ID NOs:1, 2, 5-9, and 13-21 which have from about 88% to about 99% sequence identity with SEQ ID NO:3. The invention additionally encompasses complements of SEQ ID NOs:1-3, 5-9, and 13-21. The invention additionally provides compositions comprising the cDNAs or complements thereof and a vector or a labeling moiety which may be used in methods of the invention, on a substrate, or as probes. The invention further provides a vector containing the cDNA, a host cell containing the vector, and a method for using the cDNA to make the human protein. The invention still further provides a transgenic cell line or organism comprising the vector containing a cDNA selected from SEQ ID NO:1-3, 5-9, and 13-21. In a second aspect, the invention provides a cDNA or the complement thereof which can be used in methods of detection, screening, and purification. In a further aspect, the cDNA is a single-stranded RNA or DNA molecule, a peptide nucleic acid, a branched nucleic acid, and the like.

The invention provides a method for using a cDNA to detect differential expression of a nucleic acid in a sample comprising hybridizing a cDNA to the nucleic acids, thereby forming hybridization complexes and comparing hybridization complex formation with at least one standard, wherein the comparison indicates differential expression of the cDNA in the sample. In one aspect, the method of detection further comprises amplifying the nucleic acids of the sample prior to hybridization. In another aspect, the method showing differential expression of the cDNA is used to diagnose a cancer.

The invention additionally provides a method for using a composition of the invention to screen a plurality of molecules or compounds to identify or purify at least one ligand which specifically binds the cDNA, the method comprising combining the composition with the molecules or compounds under conditions allowing specific binding, and detecting specific binding to the composition, thereby identifying or purifying a ligand which binds the composition. In one aspect, the molecules or compounds are selected from aptamers, DNA molecules, RNA molecules, peptide nucleic acids, artificial chromosome constructions, peptides, transcription factors, repressors, and regulatory molecules.

The invention provides a purified protein or a portion thereof selected from the group consisting of an amino acid sequence of SEQ ID NO:4, a variant of SEQ ID NO:4, an antigenic epitope of SEQ ID NO:4, and a biologically active portion of SEQ ID NO:4. The invention also provides a composition comprising the purified protein and a labeling moiety or a pharmaceutical carrier. The invention further provides a method of using the protein to treat a subject with cancer comprising administering to a patient i need of such treatment a composition containing the purified protein and a pharmaceutical carrier. The invention still further provides a method for using a protein to screen a library or a plurality of molecules or compounds to identify or purify at least one ligand, the method comprising combining the protein with the molecules or compounds under conditions to allow specific binding and detecting specific binding, thereby identifying or purifying a ligand which specifically binds the protein. In one aspect, the molecules or compounds are selected from DNA molecules, RNA molecules, peptide nucleic acids, peptides, proteins, mimetics, agonists, antagonists, antibodies, immunoglobulins, inhibitors, and drugs. In another aspect, the ligand is used to treat a subject with a cancer.

The invention provides a method of using a protein having the amino acid sequence of SEQ ID NO:4 to screen a plurality of antibodies to identify an antibody which specifically binds the protein comprising contacting isolated antibodies with the protein under conditions to form an antibody:protein complex, and dissociating the antibody from the protein, thereby obtaining antibody which specifically binds the protein.

The invention also provides a method of using a protein having the amino acid sequence of SEQ ID NO:4 to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing monoclonal antibodies comprises A method of using a protein to prepare a monoclonal antibody comprising immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating from culture monoclonal antibodies which specifically bind the protein.

The invention provides purified polyclonal and monoclonal antibodies which bind specifically to a protein. The invention also provides a method for using an antibody to detect expression of a protein in a sample, the method comprising combining the antibody with a sample under conditions which allow the formation of antibody:protein complexes; and detecting complex formation, wherein complex formation indicates expression of the protein in the sample. In one aspect, the amount of complex formation when compared to standards is diagnostic of cancer.

The invention provides a method for inserting a heterologous marker gene into the genomic DNA of a mammal to disrupt the expression of the endogenous polynucleotide. The invention also provides a method for using a cDNA to produce a model system, the method comprising constructing a vector containing a DNA selected from SEQ ID NO:1-3, 5-9 or 13-21, transforming the vector into an embryonic stem cell, selecting a transformed embryonic stem cell, microinjecting the transformed embryonic stem cell into a blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, model system

### BRIEF DESCRIPTION OF THE FIGURES

FIG. 1 displays the nucleotide sequence of SEQ ID NO:1 (PANA).

FIG. 2 displays the nucleotide sequence of SEQ ID NO:2 (PANC1B).

FIG. 3 displays the nucleotide alignment of SEQ ID NO:l (designated 71178), SEQ ID NO:2 (designated 20483), and SEQ ID NO:11 (designated Xs7).

FIGS. 4A and 4B display the assemblage of SEQ ID NO:1 using SEQ ID NOs:5-7 (clone numbers 71178, 180773 and 496071, respectively).

FIGS. 5A and 5B display the assemblage of SEQ ID NO:2 using SEQ ID NOs:8 and 9 (clone numbers 555403 and 020384, respectively).

FIG. 6 is a schematic illustration of the alignment among SEQ ID NOs:1, 2 and 11 (g533948;

human Xs7) with the BRCA2 locus.

FIG. 7 displays putative amino acid sequence homology between the translations of SEQ ID NOs:1 and 2 (designated prot.PANC1A and prot.PANC1B, respectively) and SEQ ID NO:10 (designated prot.xs7).

FIGS. 8A-8G display nucleotide homology between the individual nucleotide sequences (SEQ ID NOs:5-9) and the Sanger genomic sequence.

FIGS. 9A-9F show the tumor suppressor (SEQ ID NO:4)as encoded by the cDNA of SEQ ID NO:3 (which has been truncated at nucleotide 1998). The alignment was produced using MACDNASIS PRO software (Hitachi Software Engineering, South San Francisco Calif.).

FIGS. 10A and 10B show the laboratory northern analysis using SEQ ID NO:3 as a probe.

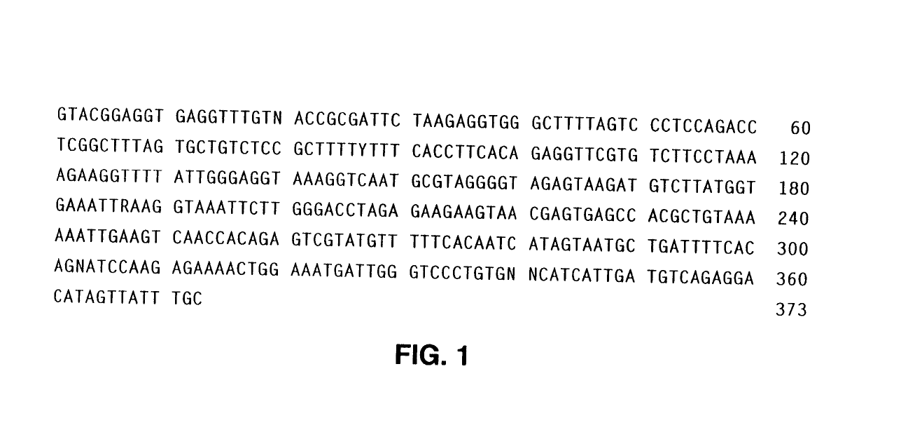

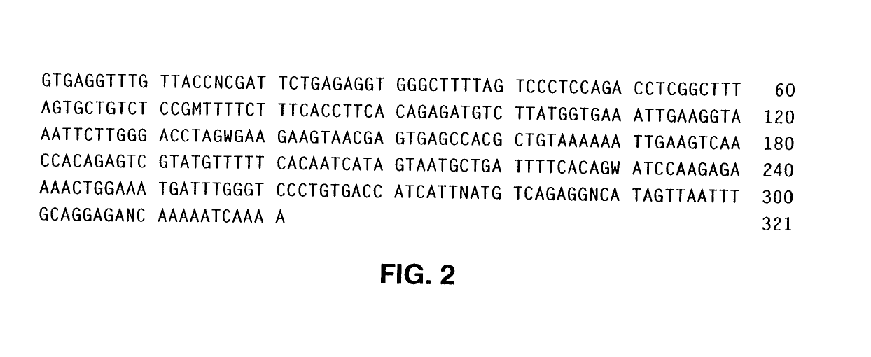

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### Definitions

“Array” refers to an ordered arrangement of at least two cDNAs or antibodies on a substrate. At least one of the cDNAs or antibodies represents a control or standard, and the other, a cDNA or antibody of diagnostic or therapeutic interest. The arrangement of two to about 40,000 cDNAs or of two to about 40,000 monoclonal or polyclonal antibodies on the substrate assures that the size and signal intensity of each labeled hybridization complex, formed between each cDNA and at least one nucleic acid, or antibody:protein complex, formed between each antibody and at least one protein to which the antibody specifically binds, is individually distinguishable.

“Tumor suppressor” refers to a purified protein obtained from any mammalian species, including bovine, canine, murine, ovine, porcine, rodent, simian, and preferably the human species, and from any source, whether natural, synthetic, semi-synthetic, or recombinant.

“Cancer” refers to cell proliferative conditions, diseases, disorders, or syndromes and specifically to human cancers of the breast, ovary, pancreas, and prostate, in which the tumor suppressor and its encoding cDNAs are differentially expressed. Differential expression of tumor suppressor is manifest by up-regulation or increase at onset of the cancer and down-regulation or absence in terminal stages of the cancer. The specific cancers which may be diagnosed using tumor suppressor include adenocarcinoma, ductal carcinoma, lobular carcinoma, and in situ carcinomas of the breast; leiomyoma, papillary serous carcinoma, mucinous cystadenoma, seroanaplastic carcinoma, and follicular cysts of the ovary; islet cell hyperplasia of the pancreas and pancreatic cancer; and adenocarcinomas of the prostate.

The “complement” of a cDNA of the Sequence Listing refers to a nucleic acid molecule which is completely complementary to the cDNA over its full length and which will hybridize to the cDNA or an mRNA under conditions of maximal stringency.

“cDNA” refers to an isolated polynucleotide, nucleic acid molecule, or any fragment or complement thereof. It may have originated recombinantly or synthetically, may be double-stranded or single-stranded, represents coding and noncoding 3′or 5′ sequence, and generally lacks introns.

A “composition” refers to the polynucleotide and a labeling moiety, a purified protein and a pharmaceutical carrier or a labeling moiety, an antibody and a labeling moiety, and the like.

“Derivative” refers to a cDNA or a protein that has been subjected to a chemical modification. Derivatization of a cDNA can involve substitution of a nontraditional base such as queosine or of an analog such as hypoxanthine. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or up-regulated or a decreased or down-regulated expression as detected by presence, absence or at least two-fold change in the amount or abundance of a transcribed messenger RNA or translated protein in a sample.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3 ′-T-C-A-G-5′. Hybridization conditions, degree of complementarity and the use of nucleotide analogs affect the efficiency and stringency of hybridization reactions.

“Labeling moiety” refers to any visible or radioactive label than can be attached to or incorporated into a cDNA or protein. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplimer, primer, and oligomer.

An “oligopeptide” is an amino acid sequence from about five residues to about 15 residues that is used as part of a fusion protein to produce an antibody.

“Portion” refers to any part of a protein used for any purpose; but especially, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR).

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Similarity” refers to the quantification (usually percentage) of nucleotide or residue matches between at least two sequences aligned using a standard algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a reproducible way to insert gaps in one of the sequences in order to optimize alignment and to achieve a more meaningful comparison between them Particularly in proteins, similarity is greater than identity in that conservative substitutions (for example, valine for leucine or isoleucine) are counted in calculating the reported percentage. Substitutions which are considered to be conservative are well known in the art.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” is a profile of gene transcription activity in a particular tissue at a particular time.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

### The Invention

The invention is based on the discovery of a tumor suppressor and its encoding cDNA. U.S. Ser. No. 08/616,392, filed Mar. 15, 1996, is incorporated herein in its entirety. The cDNA and fragments thereof, the protein and portions thereof, and an antibody which specifically binds the protein can be used directly or as compositions for the diagnosis, prognosis, treatment and evaluation of therapies for cancers, specifically of the breast, ovary, pancreas, and prostate, as defined herein.

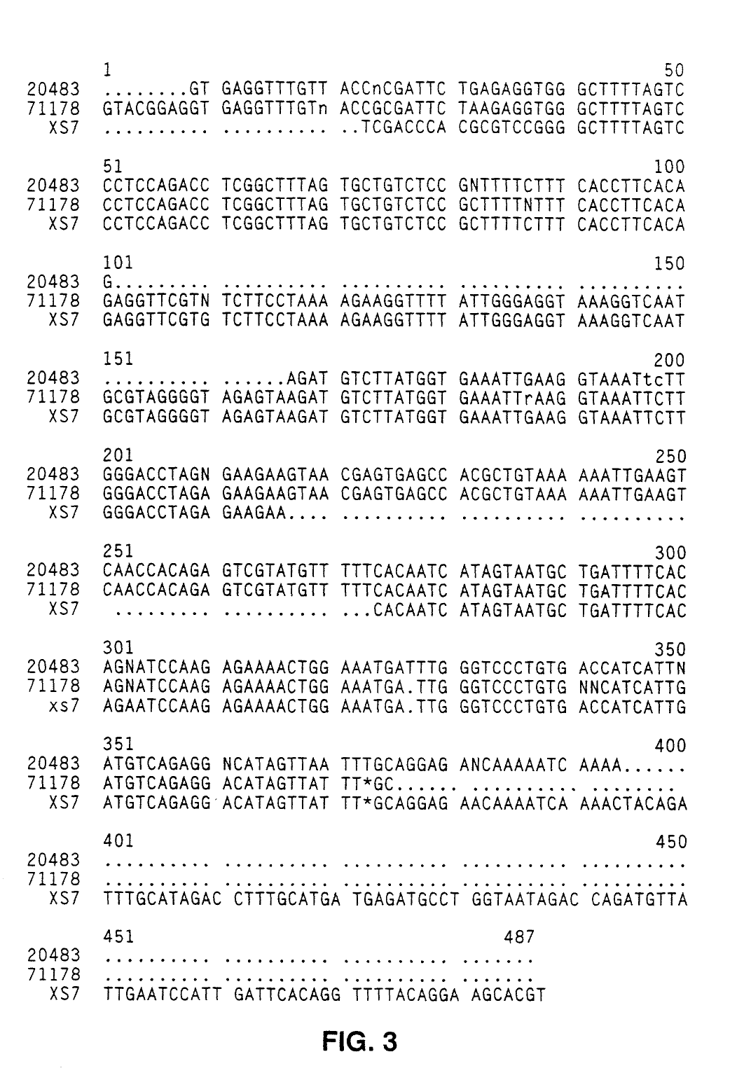

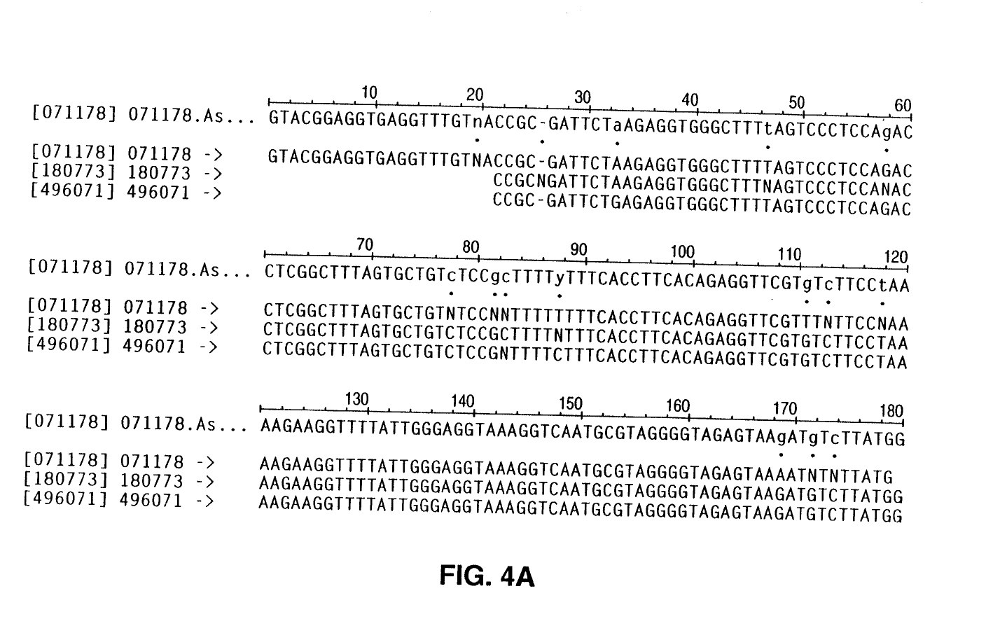

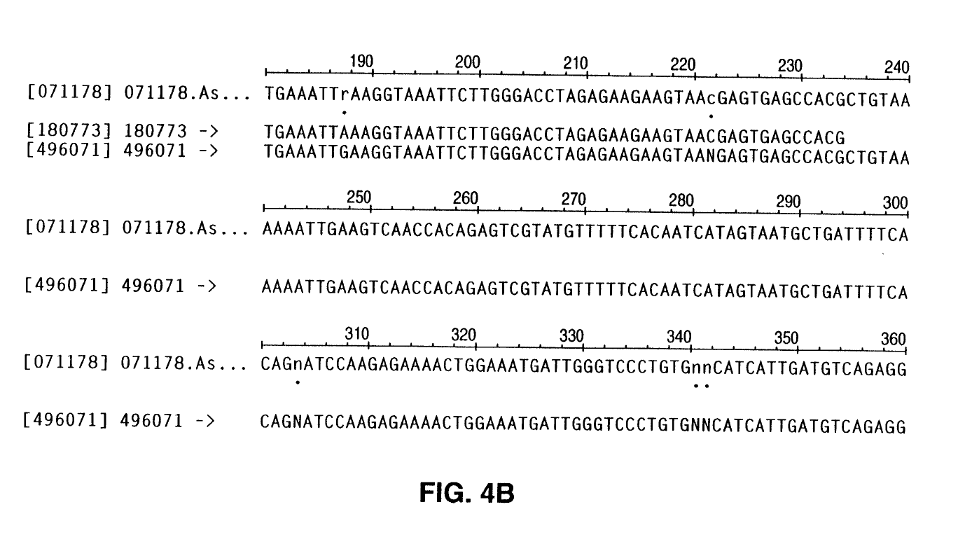

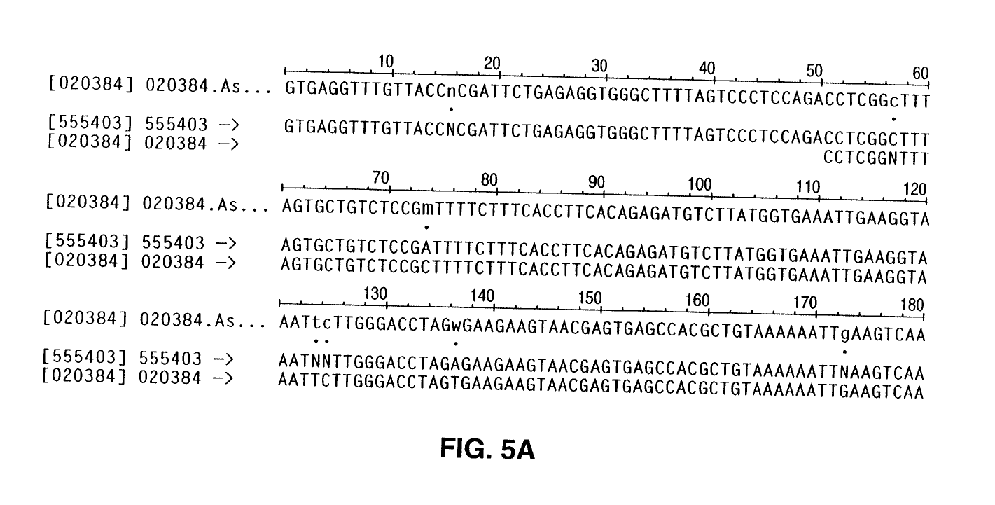

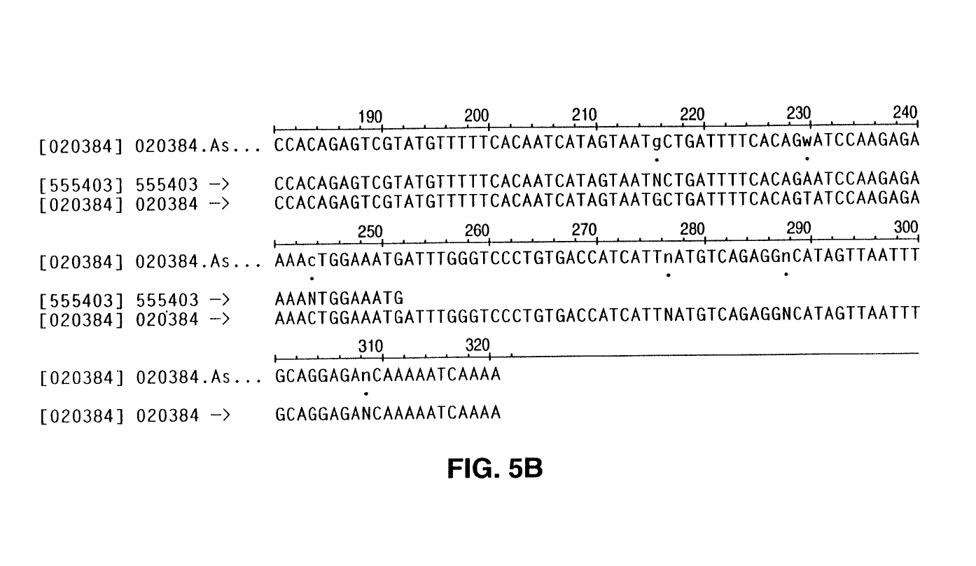

The cDNA encoding the tumor suppressor of the present invention was first identified using SEQ ID NOs:1 and 2 and the ABI Assembler of the INHERIT DNA analysis system (Applied Biosystems (ABI), Foster City Calif.). As shown in FIG. 3, SEQ ID NOs:1 and 2 were found to have homology to the nucleotide sequence of g533948 (Xs7 mRNA) which is differentially expressed in a cell line derived from pancreatic adenocarcinoma. As shown in FIGS. 4A and 4B, SEQ ID NO:1 was assembled from Incyte clones 71178, PLACNOB01; 180773 PLACNOB01; and 496071, HNT2NOT01 (SEQ ID NOs: 5-7, respectively). As shown in FIG. 5A and 5B, SEQ ID NO:2 was assembled from Incyte clone numbers 555403, SCORNOT01, and 020384, ADENINB01 (SEQ ID NOs:8 and 9, respectively). Incyte clone 496071 was used to extend and obtain the full length coding sequence, SEQ ID NO:3.

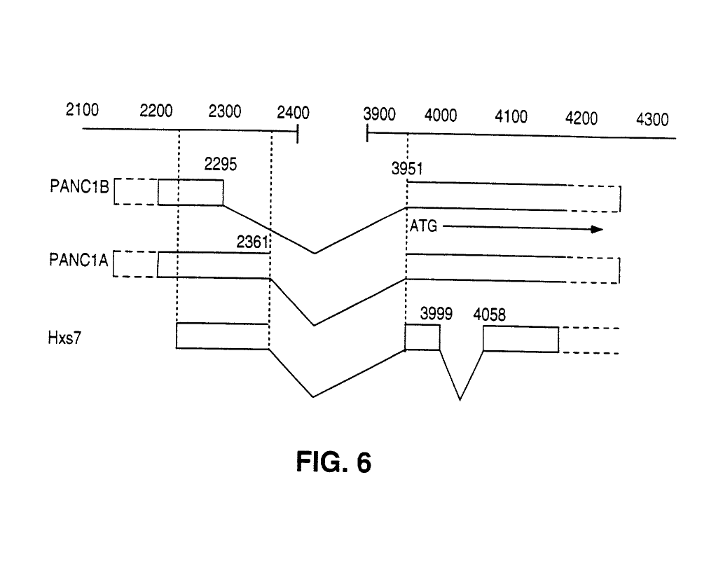

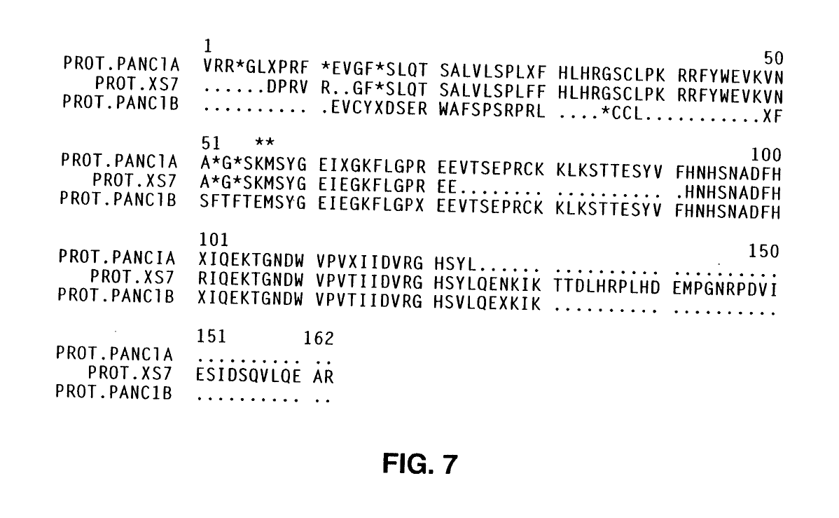

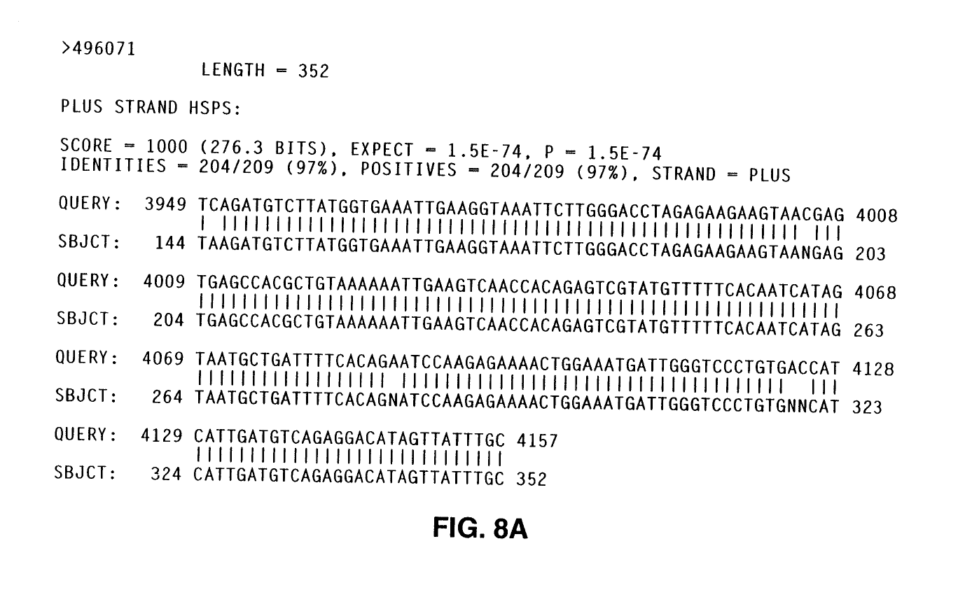

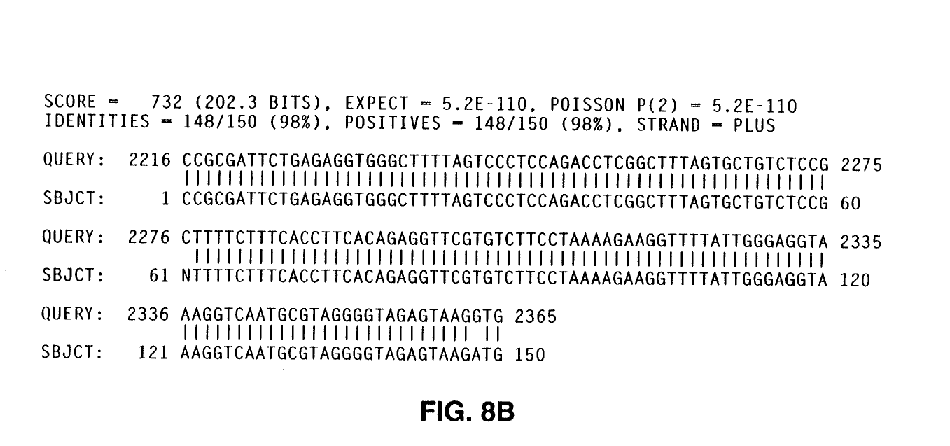

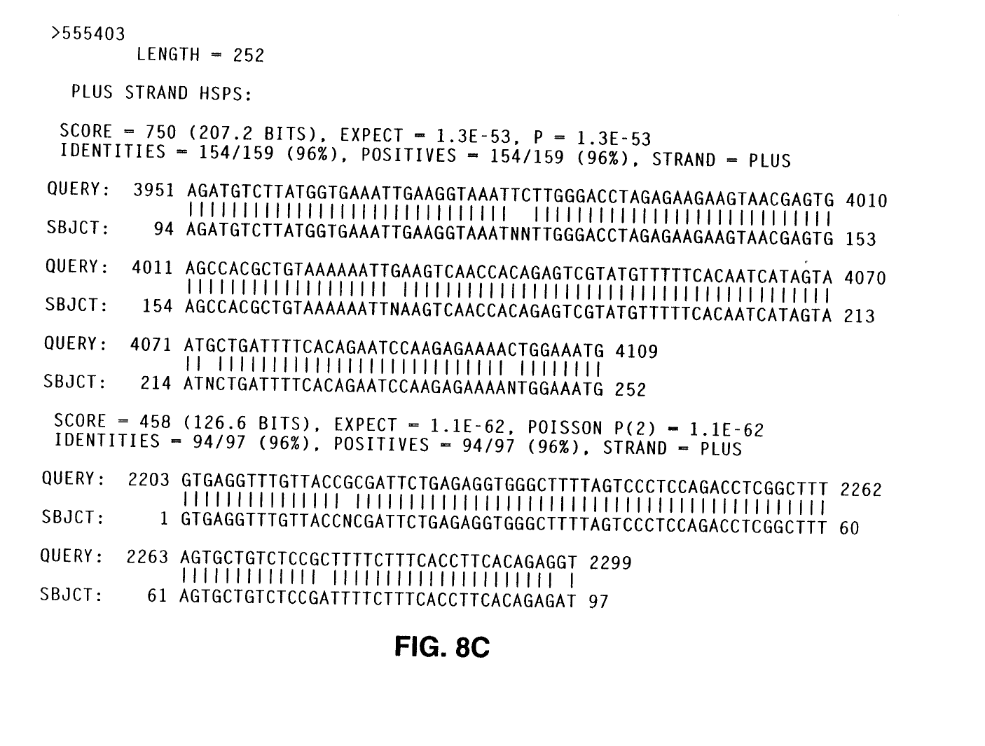

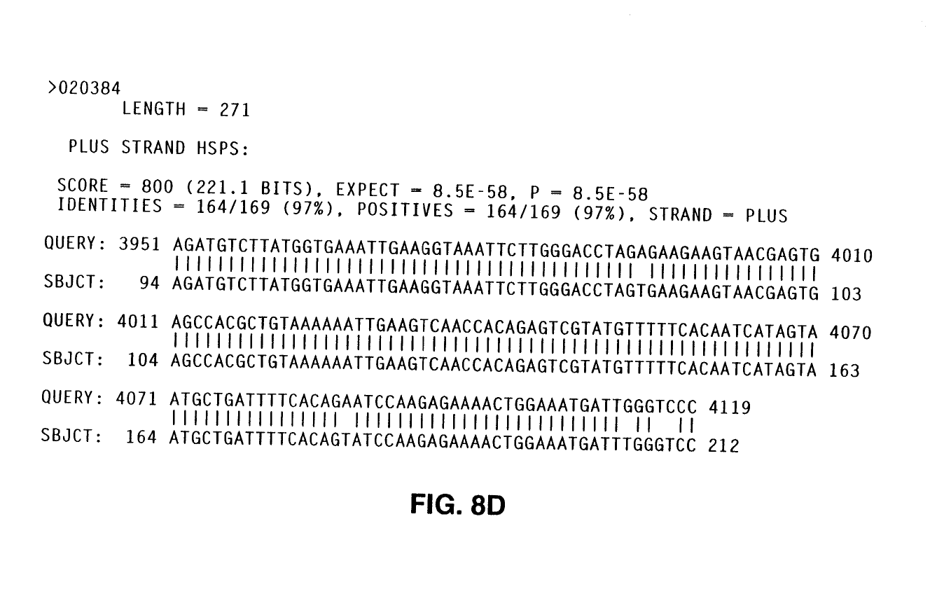

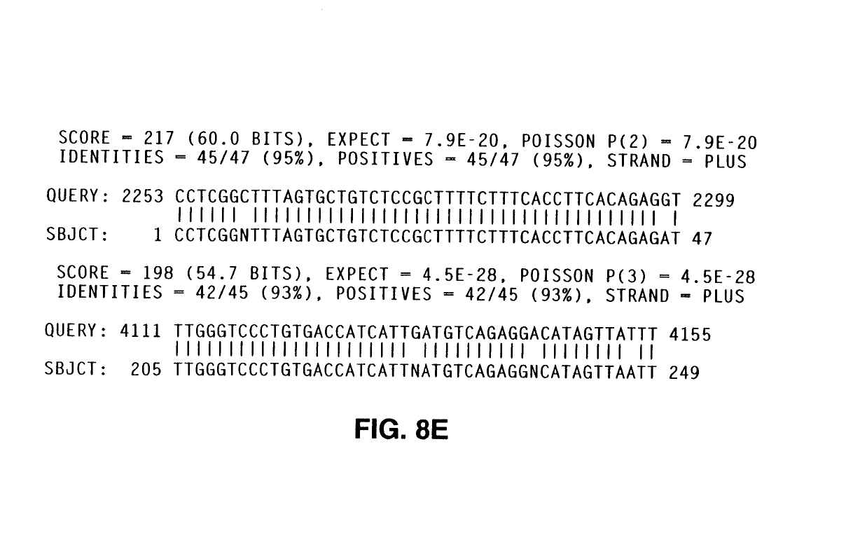

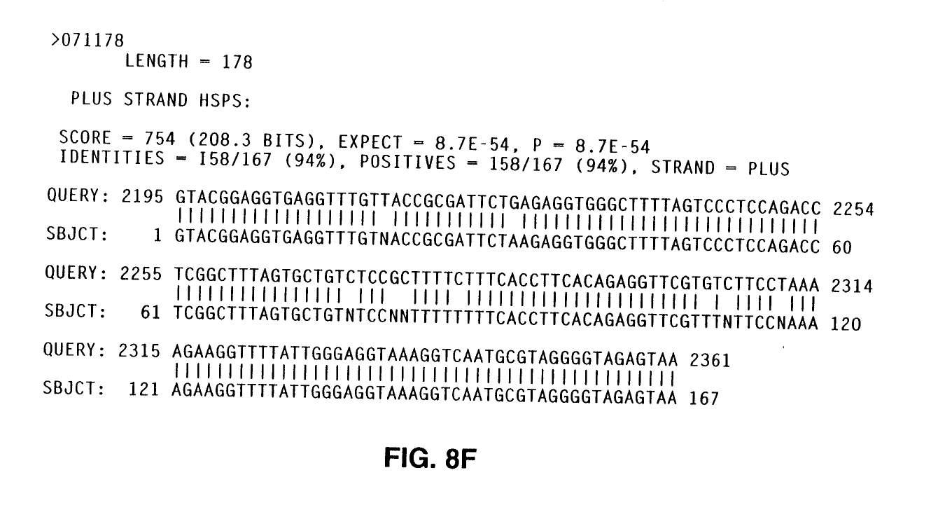

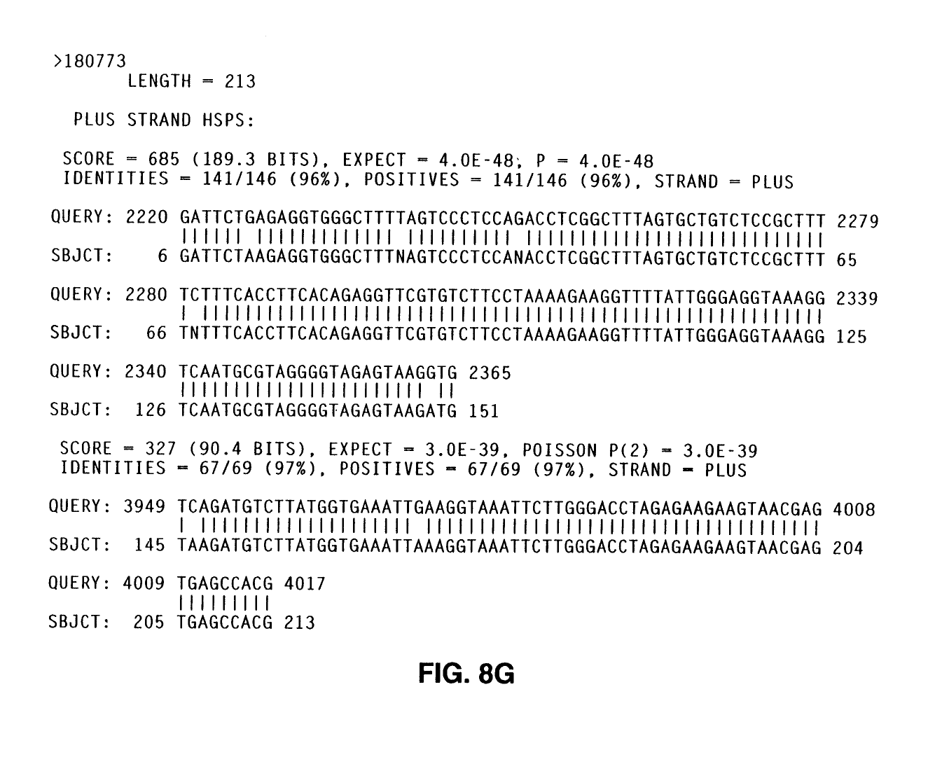

As shown schematically in FIG. 6, SEQ ID NO:1 is identical to g533948 except for an insertion of an additional 57 nucleotides between nucleotides 216 and 274 of SEQ ID NO:1; this insertion maintains the reading frame. SEQ ID NO:2 also has homology to g533948, but it has a different nucleotide sequence than SEQ ID NO:1 (FIG. 8). As shown in FIG. 7, the variation between either SEQ ID NO: 1 or SEQ ID NO:2 and human Xs7 may indicate alternative splicing events and possibly a deletion in the pancreatic cancer gene which was suggested by Schutte (supra) to be a tumor suppressor.

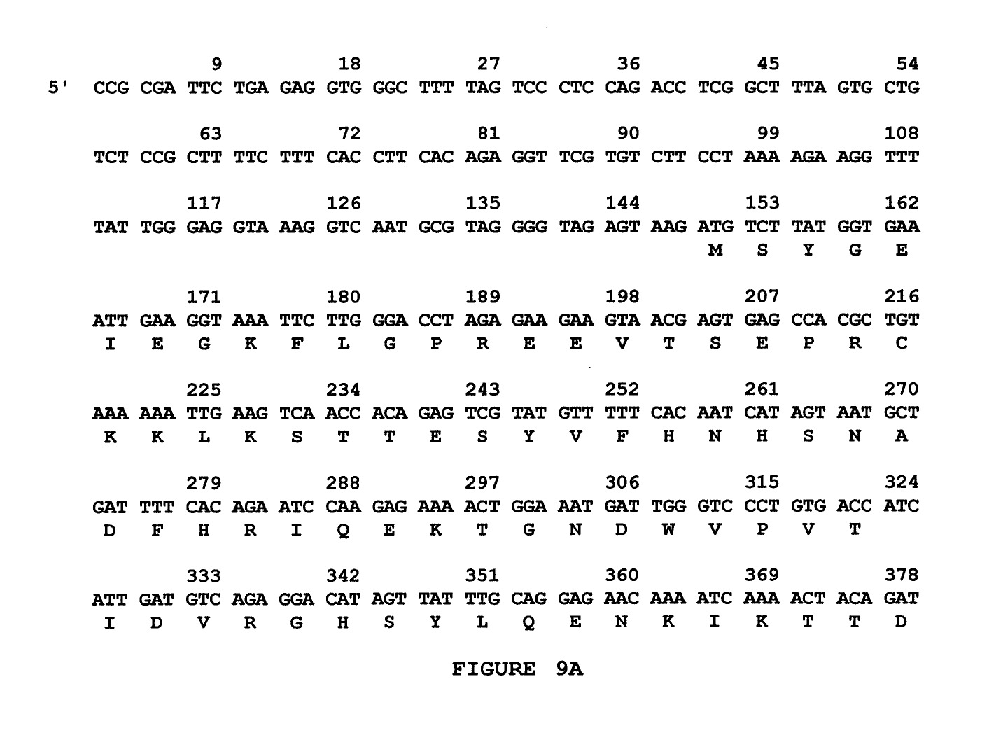

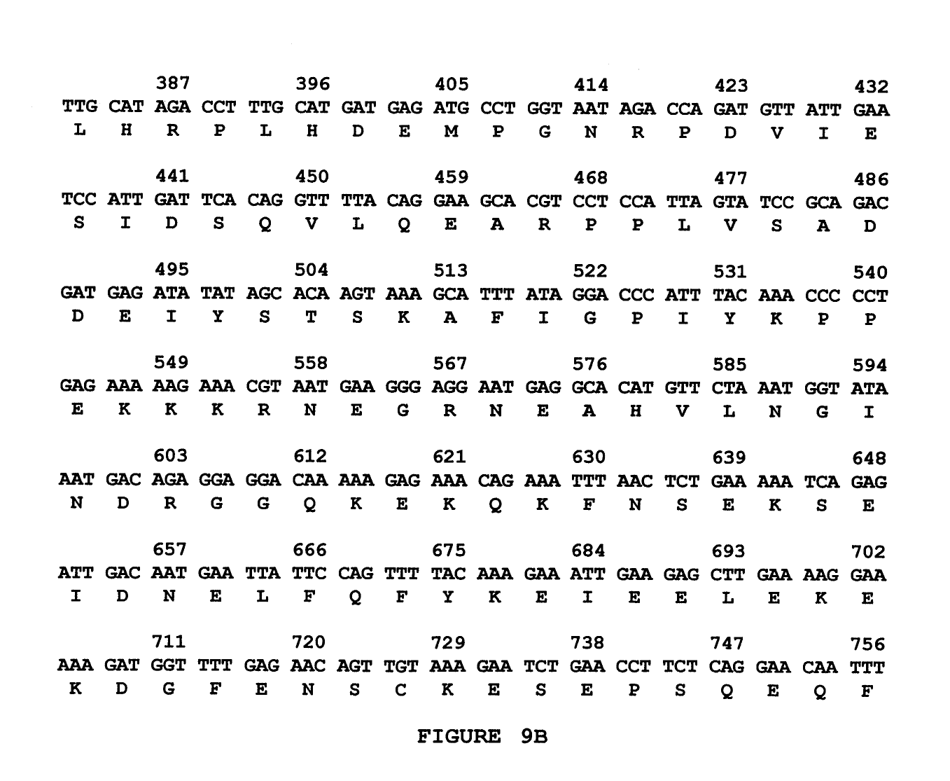

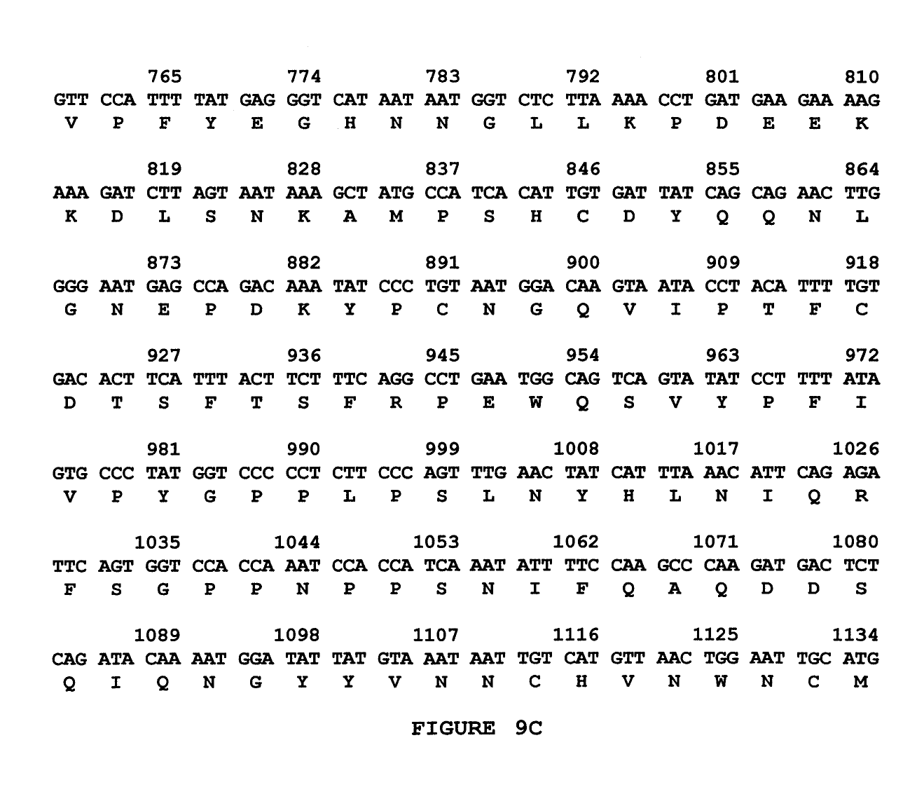

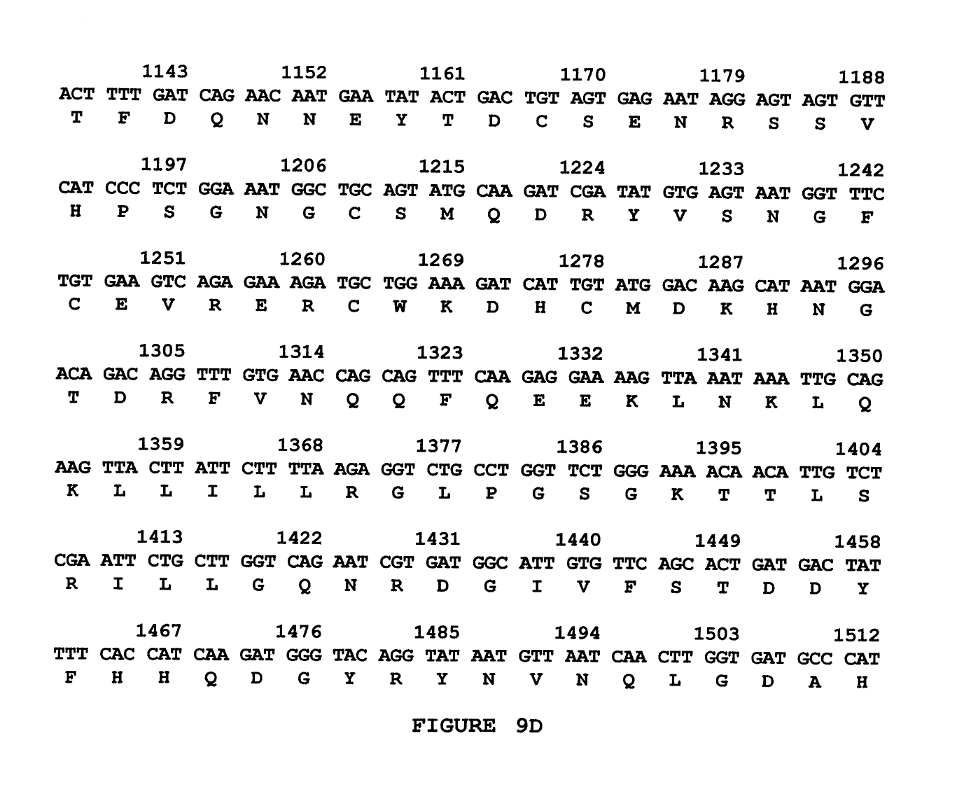

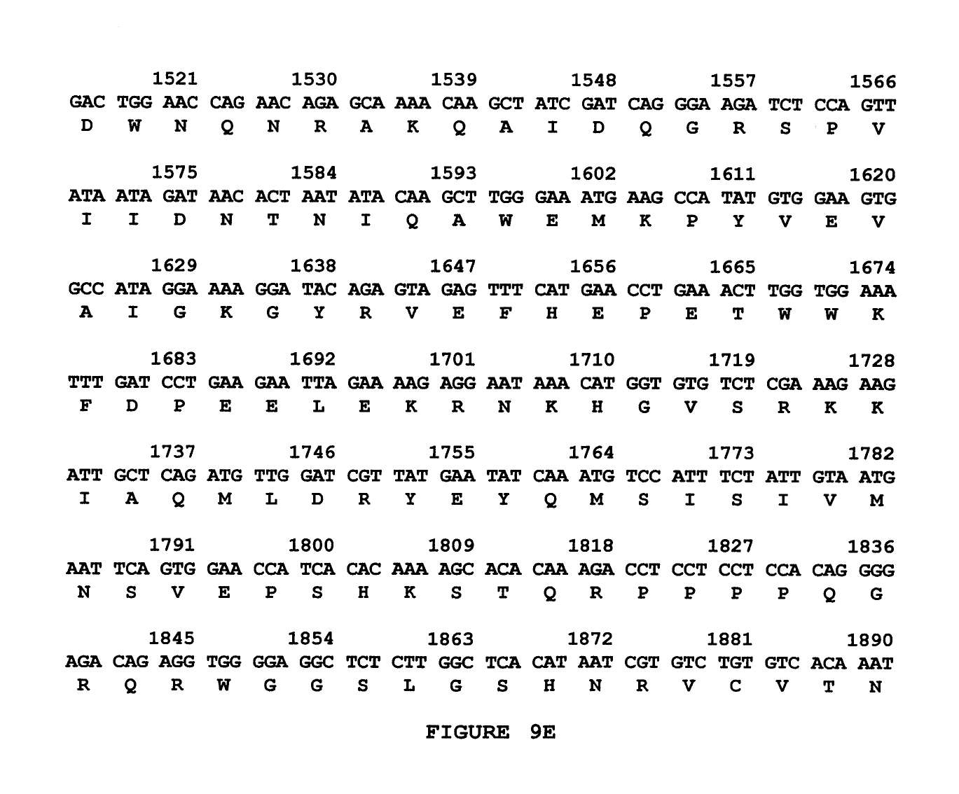

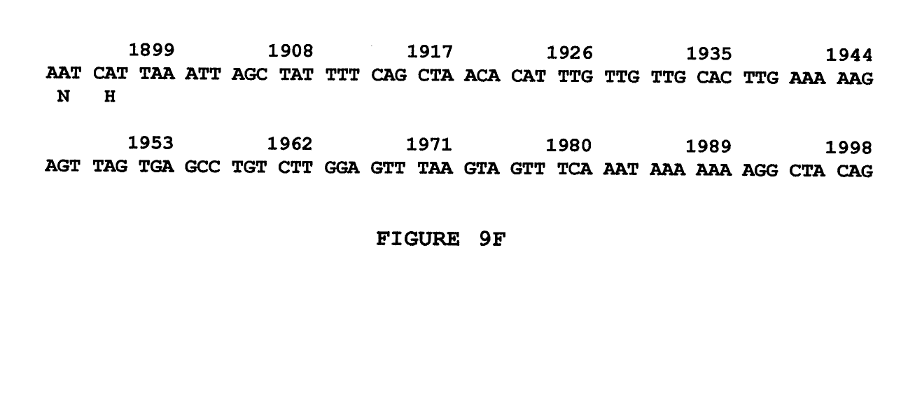

In one embodiment, the invention encompasses a protein comprising the amino acid sequence of SEQ ID NO:4 as shown in FIGS. 9A-9E. The protein is 583 amino acids in length and has three potential glycosylation sites at residues N37, N343, and N382; one cAMP- and cGMP-dependent protein kinase phosphorylation site at S123; 11 potential casein kinase II phosphorylation sites at S28, S39 T50, T58, S111, S166, S921, S231, T255, S355, and T433; 10 potential protein kinase C phosphorylation sites at T119, S163, S 192, S225, S263, T384, S413, S524, S551, and T555; and an ATP/GTP bin encompasses G₄₀₉LPGSGKT₄₁₆ of SEQ ID NO:4. BLIMPS analysis indicates that the protein has a motif that resembles a kinase from L₄₀₄-T₄₁₇. An antigenic epitope of the protein comprises residues 550-565.

The table below shows the results of microarray analysis comparing the differential expression (DE) of SEQ ID NO:4 in normal ovary relative to ovary tumor (adenocarcinoma), in BT474 ductal carcinoma cells treated with MEGM relative to untreated HMEC cells, and in DU145 prostate cancer cells treated with fibroblast growth factor (FGF) relative to starved DU145 prostate cancer cells. These results show a significant differential expression of SEQ ID NO:4 in ovary tumor and in treated BT474 and DU145 cells.

| DE (Cy5/Cy3) | Tissue Description (Cy3) | Tissue Description (Cy5) |
|---|---|---|
| −1.16 | Ovary, Nrml, mw/AdenoCA of Dn384O* | Ovary Tumor, AdenoCA, Dn3840 |
| −1.24 | Ovary, Nrml, mw/AdenoCA, Dn3840 | Ovary Tumor, AdenoCA, Dn3840 |
| −1.05 | HMEC Cells, t/starv 24 hr, Nrml | BT474 Line, t/MEGM 24 hr, Ductal CA |
| −1.06 | HMEC Cells, t/starv 24 hr, Nrml | BT474 Line, t/MEGM 24 hr, Ductal CA |
| −1.03 | DU145 Line, Untx 8 hr, Prostate CA | DU145 Line, t/FGF 50 ng/ml 8 hr, Prostate CA |

*DN3840 is the donor ID
**note that the ovary and breast experiments were run in duplicate.

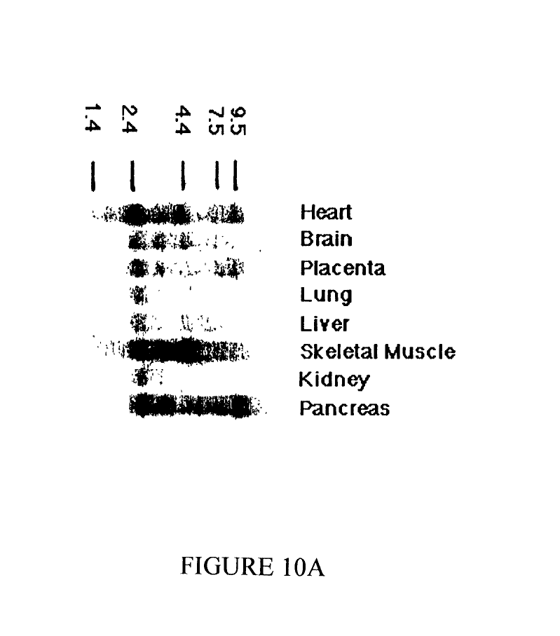

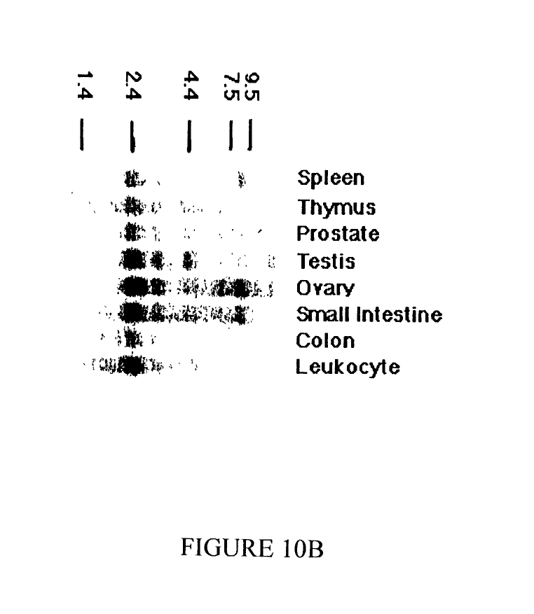

FIG. 10 shows the results of using SEQ ID NO:4 as a probe on MULTITISSUE NORTHERN BLOTS (MTN blots; Clontech, Palo Alto). This data is summarized in the second table of EXAMPLE IX.

cDNAs encoding the tumor suppressor were identified using BLAST2 with default parameters and the ZOOSEQ databases (Incyte Genonics, Palo Alto Calif.). These cDNAs have from about 88% to about 96% amino acid sequence identity to the human protein as shown in the table below. The first column shows the SEQ ID_{H} for the human cDNA; the second column, the SEQ ID_{FR} for fragment cDNAs; the third column, the sequence numbers for the fragments; the fourth column, the species; the fifth column, percent identity to the human cDNA; and the sixth column, the nucleotide alignment (Nt_{H}) of the human and variant cDNAs.

| SEQ | SEQ | | | | |
|---|---|---|---|---|---|
| ID_{H} | ID_{FR} | Clone No. | Species | Identity | Nt_{H} Alignment |
| 3 | 13 | 702456952T1 | Rat | 91% | 1400-1870 |
| 3 | 14 | 700293754F6 | Rat | 89% | 1234-1592 |
| 3 | 15 | 703193506J1 | Monkey | 96% | 1577-2065 |
| 3 | 16 | 700705495H1 | Monkey | 96% | 962-1202 |
| 3 | 17 | 703194520J1 | Monkey | 95% | 4869-5005 |
| 3 | 18 | 703518548J1 | Dog | 93% | 1426-1983 |
| 3 | 19 | 703533094J1 | Dog | 93% | 1530-1920 |
| 3 | 20 | 703523973J1 | Dog | 95% | 1388-1531 |
| 3 | 21 | 700120347H1 | Mouse | 88% | 1227-1449 |

It will be appreciated by those skilled in the art that as a result of the degeneracy of the genetic code, a multitude of cDNAs encoding tumor suppressor, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring tumor suppressor, and all such variations are to be considered as being specifically disclosed.

The cDNAs of SEQ ID NOs:1-3, 5-7, and 13-21 may be used in hybridization, amplification, and screening technologies to identify and distinguish among SEQ ID NO:3 and related molecules in a sample. The cDNAs may also be used to produce transgenic cell lines or organisms which are model systems for cancers and upon which the toxicity and efficacy of potential therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

### Characterization and Use of the Invention

### cDNA libraries

In a particular embodiment disclosed herein, mRNA is isolated from cells and tissues using methods which are well known to those skilled in the art and used to prepare the cDNA libraries. The Incyte cDNAs were isolated from cDNA libraries prepared as described in the EXAMPLES. The consensus sequences are chemically and/or electronically assembled from fragments including Incyte cDNAs and extension and/or shotgun sequences using computer programs such as PHRAP (P Green, University of Washington, Seattle Wash.), and the AUTOASSEMBLER application (ABI). After verification of the 5′ and 3′ sequence, at least one of the representative cDNAs which encode the tumor suppressor is designated a reagent. These reagent cDNAs are also used in the construction of human microarrays and are represented among the sequences on the Human Genome Gem Arrays (Incyte Genomics).

### Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech (APB), Piscataway N.J.), or combinations of polymerases and proofreading exonucleases such as those found in the ELONGASE amplification system (Life Technologies, Gaithersburg MD). Preferably, sequence preparation is automated with machines such as the MICROLAB 2200 system (Hamilton, Reno Nev.) and the DNA ENGINE thermal cycler (MJ Research, Watertown Mass.). Machines commonly used for sequencing include the ABI PRISM 3700, 377 or 373 DNA sequencing systems (ABI), the MEGABACE 1000 DNA sequencing system (APB), and the like. The sequences may be analyzed using a variety of algorithms well known in the art and described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

### Extension of a Nucleic Acid Sequence

The sequences of the invention may be extended using various PCR-based methods known in the art. For example, the XL-PCR kit (ABI), nested primers, and commercially available cDNA or genomic DNA libraries may be used to extend the nucleic acid sequence. For all PCR-based methods, primers may be designed using commercially available software to be about 22 to 30 nucleotides in length, to have a GC content of about 50% or more, and to anneal to a target molecule at temperatures from about 55 C. to about 68 C. When extending a sequence to recover regulatory elements, it is preferable to use genomic, rather than cDNA libraries.

### Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the tumor suppressor, allelic variants, or related molecules. The probe may be DNA or RNA, may be single-stranded, and should have at least 50% sequence identity to a nucleic acid sequence selected from SEQ ID NOs:1-3, 5-7, 13-21. Hybridization probes may be produced using oligolabeling, nick translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using commercially available kits.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60 C., which permits the formation of a hybridization complex between nucleic acid sequences that contain some mismatches. Subsequent washes are performed at higher stringency with buffers such as 0.2×SSC with 0.1% SDS at either 45 C. (medium stringency) or 68 C. (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St Louis Mo.) and a blocking agent such as denatured salmon sperm DNA. Selection of components and conditions for hybridization are well known to those skilled in the art and are reviewed in Ausubel (ra) and Sambrook et al. (1989) Molecular Cloning, A Laboratory Manual, Cold Spring Harbor Press, Plainview N.Y.

Arrays incorporating cDNAs or antibodies may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Monoclonal or polyclonal antibodies may be used to detect or quantify expression of a protein in a sample. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; Heller et al. (1997) U.S. Pat. No. 5,605,662; and deWildt et al. (2000) Nature Biotechnol 18:989-994.)

Hybridization probes are also useful in mapping the naturally occurring genomic sequence. The probes may be hybridized to a particular chromosome, a specific region of a chroniosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes.

### Expression

Any one of a multitude of cDNAs encoding the tumor suppressor may be cloned into a vector and used to express the protein, or portions thereof, in host cells. The nucleic acid sequence can be engineered by such methods as DNA shuffling, as described in U.S. Pat. No.5,830,721, and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mamalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

### Recovery of Proteins from Cell Culture

Heterologous moieties engineered into a vector for ease of purification include glutathione S-transferase (GST), 6xHis, FLAG, MYC, and the like. GST and 6xHis are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

### Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. These processes are described in the Novabiochem 1997/98 Catalog and Peptide Synthesis Handbook (San Diego Calif. pp.S1-S20). Automated synthesis may also be carried out on machines such as the ABI 431A peptide synthesizer (ABI). A protein or portion thereof may be purified by preparative high performance liquid chromatography and its composition confirmed by amino acid analysis or by sequencing (Creighton (1984) Proteins, Structures and Molecular Properties, WH Freeman, New York N.Y.).

### Preparation and Screening of Antibodies

Various hosts including, but not limited to, goats, rabbits, rats, mice, and human cell lines may be immunized by injection with tumor suppressor or any portion thereof. Adjuvants such as Freund's, mineral gels, and surface active substances such as lysolecithin, pluronic polyols, polyanions, peptides, oil emulsions, keyhole limpet hemacyanin (KLH), and dinitrophenol may be used to increase immunological response. The oligopeptide, peptide, or portion of protein used to induce antibodies should consist of at least about five amino acids, more preferably ten amino acids, which are identical to a portion of the natural protein. Oligopeptides may be fused with proteins such as KLH in order to produce antibodies to the chimeric molecule.

Monoclonal antibodies may be prepared using any technique which provides for the production of antibodies by continuous cell lines in culture. These include, but are not limited to, the hybridoma technique, the human B-cell hybridoma technique, and the EBV-hybridoma technique. (See, e.g., Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120.)

Alternatively, techniques described for antibody production may be adapted, using methods known in the art, to produce epitope-specific, single chain antibodies. Antibody fragments which contain specific binding sites for epitopes of the protein may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse et al. (1989) Science 246:1275-1281.)

The tumor suppressor, or a portion thereof, may be used in screening assays of phagemid or B-lymphocyte immunoglobulin libraries to identify antibodies having the desired specificity. Numerous protocols for competitive binding or immunoassays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering epitopes is preferred, but a competitive binding assay may also be employed (Pound (1998) Immunochemical Protocols, Humana Press, Totowa N.J.).

### Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Operon Technologies, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

### Diagnostics

### Nucleic Acid Assays

The cDNAs, fragments, oligonucleotides, complementary RNA and DNA molecules, and PNAs may be used to detect and quantify differential gene expression for diagnostic purposes. Cancers associated with differential expression of SEQ ID NO:3 include adenocarcinoma, ductal carcinoma, lobular carcinoma, and in situ carcinomas of the breast; leiomyoma, papillary serous carcinoma, mucinous cystadenoma, seroanaplastic carcinoma, and follicular cysts of the ovary; islet cell hyperplasia of the pancreas and pancreatic cancer; and adenocarcinomas of the prostate. The diagnostic assay may use hybridization or quantitative PCR to compare gene expression in a biological sample from a patient to standard samples in order to detect differential gene expression. Qualitative and quantitative methods for this comparison are commercially available and well known in the art.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

### Protein Assays

Detection and quantification of a protein using either labeled amino acids or specific polyclonal or monoclonal antibodies which specifically bind the protein are known in the art. Examples of such techniques include two-dimensional polyacrylamide gel electrophoresis, enzyme-linked immunosorbent assays (ELISAs), radioimmiunoassays (RIAs), and fluorescence activated cell sorting (FACS). These assays and their quantitation against purified, labeled standards are well known in the art (Ausubel, supra, unit 10.1-10.6). A two-site, monoclonal-based immunoassay utilizing monoclonal antibodies reactive to two non-interfering epitopes is preferred, but a competitive binding assay may be employed. (See, e.g., Coligan et al. (1997) Current Protocols in Immunology, Wiley-Interscience, New York N.Y.; and Pound, supra.)

### Therapeutics

As described in THE INVENTION section, chemical and structural similarity, in particular the sequences and specific motifs that exist between regions of the tumor suppressor (SEQ ID NO:4) and Xs7 (SEQ ID NO:10). In addition, differential expression was demonstrated in cancers of the breast, ovary, and prostate using microarrays. Exemplary and confirmatory transcript images for differential expression of tumor suppressor in breast, ovary, pancreatic, and prostate cancers are shown in EXAMPLE VIII. Thus, tumor suppressor clearly plays a role in multiple human cancers including, but not limited to, adenocarcinoma, ductal carcinoma, lobular carcinoma, and in situ carcinomas of the breast; leiomyoma, papillary serous carcinoma, mucinous cystadenoma, seroanaplastic carcinoma, and follicular cysts of the ovary; islet cell hyperplasia of the pancreas and pancreatic cancer; and adenocarcinomas of the prostate.

In one embodiment, a cancer which has decreased expression of the protein may be treated by the delivery of the protein or a pharmaceutical composition containing the protein. Such delivery may be effected by methods well known in the art and include delivery by an antibody specifically targeted to the cancer.

In another embodiment, the an agonist which stimulates the expression or activity of the protein may be administered to a subject to treat a cancer. In an additional embodiment, a vector expressing the cDNA which encodes tumor suppressor may be delivered into the cancer cells of a subject in need of such treatment.

Any of these compositions may be administered in combination with other therapeutic agents. Selection of the agents for use in combination therapy may be made by one of ordinary skill in the art according to conventional pharmaceutical principles. A combination of therapeutic agents may act synergistically to affect treatment of a particular cancer at a lower dosage of each agent alone.

### Modification of Gene Expression Using Nucleic Acids

Gene expression may be modified by designing complementary or antisense molecules (DNA, RNA, or PNA) to the control, 5′, 3′, or other regulatory regions of the gene encoding tumor suppressor. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule less available to endogenous endonucleases.

### Screening and Purification Assays

The cDNA encoding tumor suppressor may be used to screen a library or a plurality of molecules or compounds for specific binding affinity. The libraries may be aptamers, DNA molecules, RNA molecules, PNAs, peptides, proteins such as transcription factors, enhancers, or repressors, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the single-stranded or double-stranded molecule.

In one embodiment, the cDNA of the invention may be incubated with a plurality of purified molecules or compounds and binding activity determined by methods well known in the art, e.g., a gel-retardation assay (U.S. Pat. No. 6,010,849) or a commercially available reticulocyte lysate transcriptional assay.

In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, tumor suppressor may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulins, inhibitors, and drugs or any other ligand, which specifically binds the protein.

In one aspect, this invention comtemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a model system to evaluate their toxicity, diagnostic, or therapeutic potential.

### Pharmacology

Pharmaceutical compositions contain active ingredients in an effective amount to achieve a desired and intended purpose and a pharmaceutical carrier. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans.

A therapeutically effective dose refers to that amount of protein or inhibitor which ameliorates the symptoms or condition. Therapeutic efficacy and toxicity of such agents may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions which exhibit large therapeutic indexes are preferred. The data obtained from cell culture assays and animal studies are used in formulating a range of dosage for human use.

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

### Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted:

1) an initial dose-range-finding experiment,

2) an experiment to narrow the range of effective doses, and

3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

### Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

### Knockout Analysis

In gene knockout analysis, a region of a gene is enzymatically modified to include a non-mammalian gene such as the neomycin phosphotransferase gene (neo; Capechi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

### Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

### Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I cDNA Library Construction

The inflamed adenoid library was constructed from mixed adenoid and tonsil lymphoid tissue surgically removed from a child during a tonsilectomy. The tissue was obtained from University of California at Los Angeles and frozen for future use. The frozen tissue was ground in a mortar and pestle and lysed immediately in buffer containing guanidinium isothiocyanate (Chirgwin et al (1979) Biochemistry 18:5294). Lysis was followed by several phenol-chloroform extractions and ethanol precipitations. Poly-A+ mRNA was isolated using biotinylated oligo d(T) and streptavidin coupled to paramagnetic particles (Poly(A) Tract Isolation system; Promega).

The isolated mRNA was used by Stratagene to construct a cDNA library. cDNA synthesis was primed using oligo d(T) and/or randomhexamers. Synthetic adapter oligonucleotides were ligated onto ends of the cDNA enabling its insertion into the UNIZAP vector system (Stratagene). The quality of the cDNA library was screened using either DNA probes or antibody probes, and then the BLUESCRIPT phagemid (Stratagene) was excised. Phage particles were infected into the E. coli host strain XL1-BLUE competent cells (Stratagene).

### II Isolation of cDNA Clones

Plasmid DNA was released from the cells and purified using the MINIPREP kit (Edge Biosystems, Gaithersburg Md.). This kit consists of a 96 well block with reagents for 960 purifications. The recommended protocol was employed except for the following changes: 1) each well was filled with only 1 ml of sterile TERRIFIC BROTH (BD Biosciences, San Jose Calif.) with carbencillin (Carb) at 25 mg/l and glycerol at 0.4%; 2) the bacteria were cultured for 24 hours after the wells were inoculated and then lysed with 60 μl of lysis buffer; 3) a centrifugation step employing the GS-6R centrifuge rotor (Beckman Coulter, Fullerton Calif.) at 2900 rpm for 5 min was performed before the contents of the block were added to the primary filter plate; and 4) the optional step of adding isopropanol to TRIS buffer was not performed. After the last step in the protocol, samples were transferred to a 96-well block for storage.

III Sequencing

The cDNAs were prepared for sequencing using the MICROLAB 2200 system (Hamilton) in combination with the DNA ENGINE thermal cyclers (MJ Research). The cDNAs were sequenced by the method of Sanger and Coulson (1975; J Mol Biol 94:441-448) using an ABI PRISM 373 or 377 sequencing system (ABI). Most of the isolates were sequenced according to standard ABI protocols and kits with solution volumes of 0.25×-1.0× concentrations or using solutions and dyes from APB.

IV Extension of cDNA Sequences

The cDNA sequence was extended to full length using the Incyte clone 496071, derived from the hNT-2 cell line. A set of nested deletion sequencing templates was prepared from overnight liquid culture of clone 496071 using the ERASE-A-BASE system (Promega).

Sequencing reactions were performed with the ABI PRISM Dye Terminator cycle sequencing kit with AMPLITAQ FS DNA polymerase (ABI). PCR was performed on a DNA ENGINE thermal cycler (MJ Research). Reactions were analyzed on an ABI PRISM 310 genetic analyzer (ABI). Individual sequences were assembled and edited using ABI AutoAssembler software (ABI).

In the alternative, extension is accomplished using oligonucleotide primers synthesized to initiate 5′ and 3′ extension of the known fragment. These primers are designed using commercially available primer analysis software to be about 22 to 30 nucleotides in length, to have a GC content of about 50% or more, and to anneal to the target sequence at temperatures of about 68 C. to about 72 C. Any stretch of nucleotides that would result in hairpin structures and primer-primer dimerizations is avoided.

Selected cDNA libraries are used as templates to extend the sequence. If more than one extension is necessary, additional or nested sets of primers are designed. Preferred libraries have been size-selected to include larger cDNAs and random primed to contain more sequences with 5′ or upstream regions of genes. Genonic libraries are used to obtain regulatory elements, especially extension into the 5′ promoter binding region.

High fidelity amplification is obtained by PCR using methods such as that taught in U.S. Pat. No. 5,932,451. PCR is performed in 96-well plates using the DNA ENGINE thermal cycler (MJ Research). The reaction mix contains DNA template, 200 nmol of each primer, reaction buffer containing Mg²⁺, (NH₄)₂SO₄, and β-mercaptoethanol, Taq DNA polymerase (APB), ELONGASE enzyme (Life Technologies), and Pfu DNA polymerase (Stratagene), with the following parameters for primer pair PCI A and PCI B (Incyte Genomics): Step 1: 94 C., three min; Step 2: 94 C., 15 sec; Step 3: 60 C., one min; Step 4: 68 C., two min; Step 5: Steps 2, 3, and 4 repeated 20 times; Step 6: 68 C., five min; Step 7: storage at 4 C., In the alternative, the parameters for primer pair T7 and SK+ (Stratagene) are as follows: Step 1: 94 C., three min; Step 2: 94 C., 15 sec; Step 3: 57 C., one min; Step 4: 68 C., two min; Step 5: Steps 2, 3, and 4 repeated 20 times; Step 6: 68 C., five min; Step 7: storage at 4 C.

The concentration of DNA in each well is determined by dispensing 100 μl PICOGREEN quantitation reagent (0.25% reagent in 1×TE, v/v; Molecular Probes) and 0.5 μl of undiluted PCR product into each well of an opaque fluorimeter plate (Corning, Acton Mass.) and allowing the DNA to bind to the reagent. The plate is scanned in a Fluoroskan II (Labsystems Oy, Finland) to measure the fluorescence of the sample and to quantify the concentration of DNA. A 5 μl to 10 μL aliquot of the reaction mixture is analyzed by electrophoresis on a 1% agarose minigel to determine which reactions are successful in extending the sequence.

The extended clones are desalted, concentrated, transferred to 384-well plates, digested with CviJI cholera virus endonuclease (Molecular Biology Research, Madison Wis.), and sonicated or sheared prior to religation into pUC 18 vector (APB). For shotgun sequences, the digested nucleotide sequences are separated on low concentration (0.6 to 0.8%) agarose gels, fragments are excised, and the agar is digested with AGARACE enzyme (Promega). Extended clones are religated using T4 DNA ligase (New England Biolabs) into pUC1 8 vector (APB), treated with Pfu DNA polymerase (Stratagene) to fill-in restriction site overhangs, and transfected into E. coli competent cells. Transformed cells are selected on antibiotic-containing media, and individual colonies are picked and cultured overnight at 37 C. in 384-well plates in LB/2× carbenicillin liquid media.

The cells are lysed, and DNA is amplified using primers, Taq DNA polymerase (APB) and Pfu DNA polymerase (Stratagene) with the following parameters: Step 1: 94 C., three min; Step 2: 94 C., 15 sec; Step 3: 60 C., one min; Step 4: 72 C., two min; Step 5: steps 2, 3, and 4 repeated 29 times; Step 6: 72 min; Step 7: storage at 4 C. DNA is quantified using PICOGREEN quantitation reagent (Molecular Probes) as described above. Samples with low DNA recoveries are reamplified using the conditions described above. Samples are diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

V Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.; http://www.ncbi.nlmnih.gov/gorf/bl2.html), includes various sequence analysis programs including “blastn” that is used to align nucleotide sequences and BLAST2 that is used for direct pairwise comparison of either nucleotide or amino acid sequences. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity is measured over the entire length of a sequence. Brenner et al. (1998; Proc Natl Acad Sci 95:6073-6078, incorporated herein by reference) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfaniwustl.edu/). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VI Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding tumor suppressor that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm

VII Hybridization Technologies and Analyses

### Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH ), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

### Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below.

### Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNase inhibitor, 1 μreverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMA SPIN+TE 30 columns (Clontech). Purified probe is ethanol precipitated by diluting probe to 90 μl in DEPC-treated water, adding 2 μl 1 mg/ml glycogen, 6 μl 5 M sodium acetate, and 300 μl 100% ethanol. The probe is centrifuged for 20 min at 20,800×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

### Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester N.Y.) is exposed to the membrane overnight at −70 C., developed, and examined visually.

### Polymer Coated Slide-based Hybridization

Probe is heated to 65 C. for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of CyS. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomnics).

VIII Transcript Imaging

A transcript image was performed using the LIFESEQ GOLD database (Jun01release, Incyte Genomics). This process allowed assessment of the relative abundance of the expressed polynucleotides in all of the cDNA libraries. Criteria for transcript imaging can be selected from category, number of cDNAs per library, library description, disease indication, clinical relevance of sample, and the like.

All sequences and cDNA libraries in the LIFESEQ database have been categorized by system, organ/tissue and cell type. For each category, the number of libraries in which the sequence was expressed were counted and shown over the total number of libraries in that category. In some transcript images, all normalized or pooled libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be disregarded or removed where clinical relevance is emphasized. Conversely, fetal tissue may be emphasized wherever elucidation of inherited disorders or differentiation of particular cells or organs from stem cells (such as nerves, heart or kidney) would be furthered by removing clinical samples from the analysis.

Transcript imaging can also be used to support data from other methodologies such as microarray analysis. The transcript images for prostate, breast, and ovary below, support the microarray data shown in the table in THE INVENTION section of the application. The first column shows library name; the second column, the number of cDNAs sequenced in that library; the third column, the description of the library; the fourth column, absolute abundance of the transcript in the library; and the fifth column, percentage abundance of the transcript in the library.

The transcript image for SEQ ID NO:3 in prostate is shown below.

Category: Male Reproductive (Prostate)

| Library** | cDNAs | Description of Prostate Tissue | Abundance | % Abundance |
|---|---|---|---|---|
| PROSTUT20 | 3744 | tumor, adenoCA, 58M | 2 | 0.0534 |
| PC-0049 CIP | | | | |
| PROSBPT03 | 3784 | BPH, mw/adenoCA, 59M | 2 | 0.0529 |
| PROSNOT18 | 3915 | AH, aw/bladder TC CA, 58M | 2 | 0.0511 |
| PROSTMT07 | 3104 | AH, mw/adenoCA, 73M | 1 | 0.0322 |
| PROSTMY01 | 6460 | AR, mw/adenoCA, 55M, m/PROSTUT16 | 2 | 0.0310 |
| PROSTUT21 | 3264 | tumor, adenoCA, 61M | 1 | 0.0306 |
| PROSTUT12 | 7128 | tumor, adenoCA, 65M, m/PROSNOT20 | 2 | 0.0281 |
| PROSNOT19 | 3678 | AH, mw/adenoCA, M, m/PROSTUT13 | 1 | 0.0272 |
| PROSNOT26 | 3694 | mw/adenoCA, 65M | 1 | 0.0271 |
| PROSNOT28 | 3814 | AH, mw/adenoCA, 55M, m/PROSTUT16 | 1 | 0.0262 |
| PROSTMC01 | 3883 | AH, mw/adenoCA, 55M, m/PROSTUT16, | 1 | 0.0258 |
| PROSTUT04 | 8516 | tumor, adenoCA, 57M, m/PROSNOT06 | 2 | 0.0235 |
| PROSNOT06 | 8800 | AH, mw/adenoCA, 57M, m/PROSTUT04 | 2 | 0.0227 |
| PROSTUT05 | 6818 | tumor, adenoCA, 69M, m/PROSNOT07 | 1 | 0.0147 |
| PROSTUT09 | 11274 | tumor, TC CA, 66M, EF | 1 | 0.0089 |

**Epithelial cell line, subtracted, and normalized libraries were removed from this analysis.

In prostate, the expression of SEQ ID NO:3 is diagnostic of cancer. Expression of the tumor suppressor was seen as expected in early stage cancer or in cytologically normal tissues which are matched with or associated with cancer. Expression was not seen in 24, cytologically normal, prostate libraries (PROSBPS05, PROSBPT02, PROSBPT06, PROSBPT07, PROSDIP01, PROSDIP02, PROSDIP03, PROSDIT01, PROSNOP01, PROSNOP03, PROSNOT01, PROSNOT02, PROSNOT05, PROSNOT11, PROSNOT14, PROSNOT15, PROSNOT16, PROSTMC02, PROSTME06, PROSTMF03, PROSTMT01, PROSTMT02, PROSTMT03, and PROSTMT05).

The transcript image for SEQ ID NO:3 in breast is shown below.

Category: Exocrine (Breast)

| Library** | cDNAs | Description of Tissue | Abundance | % Abundance |
|---|---|---|---|---|
| BRSTNOT01* | 4627 | breast, medical history unknown, 56F | 6 | 0.1297 |
| BRSTDIT01 | 3394 | PF changes, mw/intraductal cancer, 48F | 3 | 0.0884 |
| BRSTTUT08 | 3928 | tumor, adenoCA, 45F, m/BRSTNOT09 | 2 | 0.0509 |
| BRSTNOT12 | 4182 | NF breast disease, 32F | 2 | 0.0478 |
| BRSTTUT02 | 7066 | tumor, adenoCA, 54F, m/BRSTNOT03 | 3 | 0.0425 |
| BRSTTUT17 | 2690 | tumor, ductal CA, 65F | 1 | 0.0372 |
| BRSTNOR01 | 3107 | breast, mw/lobular CA, BRSTTUT22, 59F | 1 | 0.0322 |
| BRSTTMT02 | 3240 | PF changes, mw/multifoc ductal CA, 46F | 1 | 0.0309 |
| BRSTTUT03 | 10092 | tumor, lobular CA, 58F, m/BRSTNOT05 | 3 | 0.0297 |
| BRSTNOT28 | 3734 | PF changes, 40F | 1 | 0.0268 |
| BRSTTUT18 | 3736 | tumor, ductal CA, 68F | 1 | 0.0268 |
| BRSTNOT32 | 3766 | NF breast disease, 46F | 1 | 0.0266 |
| BRSTTUT22 | 3774 | tumor, lobular CA, 59F, m/BRSTNOT16 | 1 | 0.0265 |
| BRSTNOT14 | 3792 | breast, mw/ductal CA BRSTTUT14, 62F | 1 | 0.0264 |
| BRSTNOT13 | 3860 | breast, mw/neoplasm, 36F | 1 | 0.0259 |
| BRSTNOT23 | 4056 | NF breast disease, 35F | 1 | 0.0247 |
| BRSTNOT24 | 4413 | breast, NF breast disease, 46F | 1 | 0.0227 |
| BRSTNOT04 | 10316 | breast, mw/ductal CA, aw/node mets, 62F | 2 | 0.0194 |
| BRSTTUT01 | 10643 | tumor, adenoCA, 55F, m/BRSTNOT02 | 2 | 0.0188 |
| BRSTTUT15 | 6535 | tumor, adenoCA, 46F, m/BRSTNOT17 | 1 | 0.0153 |
| BRSTNOT07 | 10055 | breast, mw/adenoCA, intraductal CA, 43F | 1 | 0.0099 |
| BRSTNOT05 | 13205 | breast, mw/lobular CA BRSTTUT03, 58F | 1 | 0.0076 |

*Victim of motor vehicle accident, no medical history available.
**Libraries made from cell lines and from normalized tissues were removed from this analysis.

In breast, expression SEQ ID NO:3 is diagnostic of cancer. Expression of SEQ ID NO:3 was not seen in cytologically normal breast libraries, BRSTNOT25 and BRSTNOT35, made from tissues removed during surgery for bilateral breast reduction.

Category: Female Reproductive (Ovary)

| Library** | cDNAs | Description of Tissue | Abundance | % Abundance |
|---|---|---|---|---|
| OVARDIT01 | 3800 | endometriosis, aw/leiomyomata, 39F | 3 | 0.0789 |
| OVARTUM02 | 2932 | tumor, papillary serous CA, 64F, WM/WN | 2 | 0.0682 |
| OVARTUT02 | 3532 | tumor, mucinous cystadenoma, 51F | 2 | 0.0566 |
| OVARTUT03 | 4246 | tumor, seroanaplastic CA, 52F | 2 | 0.0471 |
| OVARNOT09 | 4284 | follicular cysts, 28F | 2 | 0.0467 |
| OVARTUT01 | 11450 | mucinous cystadenoCA, 43F, m/OVARNOT03 | 4 | 0.0349 |

**Libraries made from normalized or subtracted tissues were removed from this analysis.

In ovary, SEQ ID NO:3 is diagnostic of cancer. SEQ ID NO:3 was not expressed in nine cytologically normal ovary libraries (OVAENOPO0,1 OVARNOM0,1, OVARNON01, OVARNON03, OVARNOP0, OVARNOT03, OVARNOT11, OVARNOT12, and OVASNOP01) or in libraries made from tissues of patients diagnosed with stromal hyperthecosis (OVARDIR01) or endometriosis (OVARDIT05 and OVARDIT06).

The transcript image for SEQ ID NO:3 in pancreas is shown below.

Category: Pancreas

| Library** | cDNAs | Description of Tissue | Abundance | % Abundance |
|---|---|---|---|---|
| PANCNOT15 | 3639 | pancreas, islet cell hyperplasia, 15M | 1 | 0.0275 |
| PANCTUT02 | 11545 | pancreatic tumor, anaplastic CA, 45F | 1 | 0.0087 |

**Libraries made from fetal tissues were removed from this analysis.

In pancreas, SEQ ID NO:3 is diagnostic of cancer. It was never expressed in seven adult, cytologically normal pancreas libraries (PANCNON03, PANCNOP03, PANCNOP05, PANCNOT01, PANCNOT08, PANCNOT17, and PANCNOT22) or in pancreas libraries from adults diagnosed with pancreatitis (PANCNOT08) or type II diabetes (PANCDIT03).

In assays using normal and cancerous standards and patient samples, the cDNA, an mRNA, tumor suppressor or an antibody specifically binding the protein serves a clinically relevant diagnostic marker for breast, ovary, pancreas, or prostate cancer.

IX Northern Analysis

Incyte clone 496071 was compared with all the other sequences in the LIFESEQ database (Incyte Genomnics, Palo Alto Calif.) using BLAST analysis Altschul (1993 and 1990) supra).

The results of the BLAST comparison in the LIFESEQ database (Incyte Genomics) using the full length insert of clone 496071 (5035 bases) as the query sequence at the time of filing is shown in the table below. SEQ ID NO:3 was expressed in PROSTUT05, BRSTTUT02, and BRSTNOT01 libraries. The

| INCYTE CLONE NO. | LIBRARY NAME | TISSUE SOURCE |
|---|---|---|
| 496071 | HNT2NOT01 | hNT-2 cell line |
| 002634 | HMC1NOT01 | HMC-1 mast cell line |
| 416343 | BRSTNOT01 | Breast |
| 569172 | MMLR3DT01 | Macrophages |
| 410543 | BRSTNOT01 | Breast |
| 533756 | BRAINOT03 | Brain |
| 020384 | ADENINB01 | Inflamed Adenoid |
| 811635 | LUNGNOT04 | Lung |
| 555403 | SCORNOT01 | Spinal Cord |
| 413301 | BRSTNOT01 | Breast |
| 419967 | BRSTNOT01 | Breast |
| 547068 | BEPINOT01 | Bronchial Epithelium |
| 346962 | THYMNOT02 | Thymus |
| 180773 | PLACNOB01 | Placenta |
| 624486 | PGANNOT01 | Brain, paraganglia |
| 497695 | NEUTLPT01 | Granulocytes treated with |
| | | LPS |
| 411476 | BRSTNOT01 | Breast |
| 413186 | BRSTNOT01 | Breast |
| 358124 | SYNORAB01 | Rheumatoid synovium, |
| | | hip |
| 570027 | MMLR3DT01 | Macrophages |
| 256001 | HNT2RAT01 | hNT cell line treated with |
| | | retinoic acid |
| 415897 | BRSTNOT01 | Breast |
| 839657 | PROSTUT05 | Prostate tumor |
| 472797 | MMLR1DT01 | Macrophages |
| 683486 | UTRSNOT02 | Uterus |
| 494487 | HNT2NOT01 | hNT cell line |
| 071178 | PLACNOB01 | Placenta |
| 043863 | TBLYNOT01 | Leukemic T&B |
| | | lymphoblasts |
| 644148 | BRSTTUT02 | Breast Tumor |
| 347204 | THYMNOT02 | Thymus |
| 391756 | TMLR2DT01 | Lymphocytes |

Incyte clone 496071 was also to screen northern blots. A DNA probe was generated by EcoRI digistion of the plasmid containing the cDNA probe was fractuionated on a 1% agarose gel and the 1.4 kb restriction fragment was exicise from the gel and purified of a QIAQUICK column (Qiagen). The 1.4 kb fragments extends from bases 1 of SEQ ID NO:3 and is comprised of the 5′untranslated region and most of the coding sequence. The probe was prepared by random priming using the REDIPRIME labeling kit with REDIVUE[−³²P]d-CTP (3000 Ci/mmol) both purchase from Amersham Pharmacia Biotech. unincorporated radioactivity was removed by column chromatography using a SEPHADEX G-50 NICK column purchased from Amersham Pharmacia Biotech.

MTN-I and MTN-II blots were purchased from Clontech. Each blot contains approximately 2 ug of poly A+per lane from various tissues. RNA was electrophoresed on a denaturing fomaldehyde 1.2% agarose gel, blotted on a nylon membrane and fixed by UV irradiation. RAPID-HYB hybridization buffer was purchased from Amersham Pharmacia Biotech.

Blots were pre-hybridized for 1 hour at 65 C. Hybridizations were performed at 65 C. using 0.5×10 cpm/ml probe for 1 hour. Blots were washed for 2×10 minutes in 1×SSC, 0.1% SDS at room temperature followed by 2 stringent washes at 65 C. in 0.1×SSC, 0.2% SDS for 10 minutes each. Blots were wrapped in SARAN WRAP plastic film (Dow Chemical, Midland Mich.) and autoradiographed at −70 C. using 2 intensifying screens and HYPERFILM-MP (Amersham Pharmacia Biotech).

The northern blots prepared using Incyte clone 496071 as a probe are shown in FIG. 10. The results showed multiple transcripts in all 16 of the tissues represented. The sizes of the transcripts were: 2.4, 3, 4.4, 9.5 and 11 kb. The major transcripts were 2.4, 4.4 and 9.5 kb. These were most abundant in skeletal muscle, pancreas, small intestine, ovary and testis. The transcript abundance varied from tissue to tissue. For example, the 2.4 kb transcript is most abundant in the majority of the tissues; however, the 4.4 kb band is more abundant than the 2.4 kb band in skeletal muscle. In addition, the 9.5 kb band is more abundant in pancreas than in the other tissues tested.

| TISSUE | 2.4 kb | 3 kb | 4.4 kb | 9.5 kb | 11 kb |
|---|---|---|---|---|---|
| Heart | X | X | X | X | |
| Brain | X | X | X | X | |
| Placenta | X | X | X | X | |
| Lung | X | X | X | | |
| Liver | X | | X | X | |
| Sk. muscle | X | X | X | X | |
| Kidney | X | X | X | | |
| Pancreas | X | X | X | X | X |
| Spleen | X | X | X | X | X |
| Thymus | X | X | X | X | X |
| Prostate | X | X | X | X | X |
| Testis | X | X | X | X | X |
| Ovary | X | X | X | X | X |
| Small | X | X | X | X | X |
| intestine | | | | | |
| Colon | X | X | | | |
| Leukocyte | X | X | X | X | |

### X Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system. Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

XI Expression of Tumor Suppressor

Expression and purification of the protein are achieved using either a mammalian or an insect cell expression system. The pUB6/V5-His vector system (Invitrogen, Carlsbad Calif.) is used to express tumor suppressor in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6xHis) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6xhis which enables purification as described above. Purified protein is used in the following activity and to make antibodies.

XII Production of Antibodies

Tumor suppressor is purified using polyacrylamide gel electrophoresis and used to immunize mice or rabbits. Antibodies are produced using the protocols well known in the art and summarized below. Alternatively, the amino acid sequence of tumor suppressor is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an 431A peptide synthesizer (ABI) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories, Palo Alto Calif.), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/l 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (−Leu). Interaction also activates expression of B-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

XVI Demonstration of Tumor Suppressor Activity

An in vitro assay for tumor suppressor activity measures the transformation of normal human fibroblast cells overexpressing antisense RNA (Garkavtsev, I. et al. (1996) Nat. Genet. 14:415-420). The cDNA of SEQ ID NO:3 is subcloned into the pLNCX retroviral vector to enable expression of antisense RNA. The resulting construct is transfected into the ecotropic BOSC23 virus-packaging cell line. Virus contained in the BOSC23 culture supernatant is used to infect the amphotropic CAK8 virus-packaging cell line. Virus contained in the CAK8 culture supernatant is used to infect normal human fibroblast (Hs68) cells. Infected cells are assessed for the following quantifiable properties characteristic of transformed cells: growth in culture to high density associated with loss of contact inhibition, growth in suspension or in soft agar, formation of colonies or foci, lowered serum requirements, and ability to induce tumors when injected into immunodeficient mice. The activity of tumor suppressor is proportional to the extent of transformation of Hs68 cells.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A purified protein comprising an amino acid sequence selected from the group consisting
a) an amino acid sequence of SEQ ID NO:4; and
b) an amino acid sequence having 83% identity to SEQ ID NO:4.

2. An antigenic epitope of the protein of claim 1 comprising residues 550 through 565 of SEQ ID NO:4.

3. A biologically active portion of the protein of claim 1 consisting of residues 404 through 417 of SEQ ID NO:4.

4. A composition comprising the protein of claim 1 and a labeling moiety or a pharmaceutical carrier.

5. A method for using a protein to screen a plurality of molecules or compounds to identify at least one ligand, the method comprising:
a) combining the protein of claim 1 with the molecules or compounds under conditions to allow specific binding; and
b) detecting specific binding, thereby identifying a ligand which specifically binds the protein.

6. The method of claim 5 wherein the molecules or compounds are selected from DNA molecules, RNA molecules, peptide nucleic acids, peptides, proteins, mimetics, agonists, antagonists, antibodies, immunoglobulins, inhibitors, and drugs.

7. A method of using a protein to prepare and purify a polyclonal antibody comprising:
a) immunizing a animal with the protein of claim 1 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

8. A method of using a protein to prepare a monoclonal antibody comprising:
a) immunizing a animal with the protein of claim 1 under conditions to elicit an antibody response;
b) isolating antibody producing cells from the animal;
c) fusing the antibody producing cells with immortalized cells in culture to form monoclonal antibody producing hybridoma cells;
d) culturing the hybridoma cells; and
e) isolating from culture monoclonal antibodies which specifically bind the protein.

9. An agonist identified by the method of claim 6

10. A polyclonal antibody produced by the method of claim 7.

11. A monoclonal antibody produced by the method of claim 8.

12. A method for using an antibody to detect expression of a protein in a sample, the method comprising:
a) combining the polyclonal antibody of claim 10 with a sample under conditions which allow the formation of antibody:protein complexes; and
b) detecting complex formation, wherein complex formation indicates expression of the protein in the sample.

13. The method of claim 12 wherein complex formation is compared with standards and is diagnostic of cancer.

14. A method for using an antibody to detect expression of a protein in a sample, the method comprising:
a) combining the monoclonal antibody of claim 11 with a sample under conditions which allow the formation of antibody:protein complexes; and
b) detecting complex formation, wherein complex formation indicates expression of the protein in the sample.

15. The method of claim 14 wherein complex formation is compared with standards and is diagnostic of cancer.
